(19) United States
(12) Patent Application Publication
(10) Pub. No.: US 2026/0077132 A1
(43) Pub. Date: Mar. 19, 2026

(54) POWER ASSIST FOR A MANUAL AND AUTOINJECTOR INJECTION DEVICE

(71) Applicant: Janssen Biotech, Inc., Horsham, PA (US)

(72) Inventors: Emma Louise Hubert, San Jose, CA (US); Jingli Wang, San Jose, CA (US); Francesco N. Albertini, Zurich (CH); Monica A. Kapil, San Jose, CA (US)

(21) Appl. No.: 19/109,250

(22) PCT Filed: Sep. 26, 2023
(86) PCT No.: PCT/IB2023/059485

§ 371 Date: Mar. 6, 2025

Related U.S. Application Data
(60) Provisional application No. 63/412,010, filed on Sep. 30, 2022.

(51) Int. Cl. A61M 5/315; A61M 5/20; A61M 5/32

(52) U.S. Cl. CPC A61M 5/31505; A61M 5/2033; A61M 5/3243; A61M 2005/2026; A61M 2005/2073

(57) ABSTRACT

A power-assisted injection device can administer a liquid medication. The injection device can include a syringe, a housing, and a motive force device. The syringe can include a plunger and a barrel configured for holding the liquid medication. The housing can include a body configured for holding the syringe. The housing can also include a latch configured to define a locked position that inhibits movement of the plunger and a released position that enables movement of the plunger. The motive force device can be configured for applying an injection force to the plunger to administer the medication while the latch is in the released position. The injection device can be configured for administering the liquid medication upon release of the latch from the locked position at least in part via the motive force device applying the injection force to the plunger of the syringe.

### BACKGROUND

Subcutaneous injections are commonly used to deliver drug to patients using various injection devices such as syringes, needle safety devices, and various autoinjectors. Examples of needle safety devices includes UltraSafe Injection System and UltraSafe Plus Injection System, marketed by Becton, Dickinson and Company.

Typical needle safety devices often require the user to remove the cap, insert the exposed needle at an injection site, and manually push the plunger with their thumb until the injection is complete. Some injectors then retract the needle and empty syringe into the body of the device. The device may then be disposed.

Existing autoinjectors can require a user to remove the cap, place the device (with needle hidden by a needle shield) against the skin and push down firmly. The start of the delivery is triggered by pushing a button or by depressing the needle guard. The user continues to hold the device against the skin until the delivery is completed. To deliver a drug with high viscosity and/or high volume, the user can be required to exert more force and/or to inject for a greater period of time and frequency (relative to less viscous and/or lower volume). In any of these circumstances, the user may experience hand discomfort or fatigue. Therefore, an improved injection device is desired.

### SUMMARY

A power-assisted injection device can administer a liquid medication. The injection device can include a syringe, a housing, and a motive force device. The syringe can include a plunger and a barrel configured for holding the liquid medication. The housing can include a body configured for holding the syringe. The housing can also include a latch configured to define a locked position that inhibits movement of the plunger and a released position that enables movement of the plunger. The motive force device can be configured for applying an injection force to the plunger to administer the medication while the latch is in the released position. The injection device can be configured for administering the liquid medication upon release of the latch from the locked position at least in part via the motive force device applying the injection force to the plunger of the syringe.

A power-assisted injection device for administering a liquid medication can include a syringe, at least one spring, a housing, and a load actuator. The syringe can include a plunger and a barrel that is configured for holding the liquid medication. The at least one spring can be configured for applying an injection force to the plunger to administer the medication. The housing can include a body configured for holding the syringe and a flange configured to translate relative to the housing body and configured to contact the plunger to apply the injection force to the syringe. The load actuator can be configured for loading the spring. The injection device can be configured for administering the liquid medication upon loading of the spring.

A power-assisted injection device for administering a liquid medication can include a syringe, a housing, a barb assembly, a plenum, and a compressed gas canister. The syringe can include a barrel that is configured for holding the liquid medication and a piston configured for administering the medication. The housing can include a body configured for holding the syringe. The barb assembly can be located within the housing and can include a barb and a support structure configured to support the barb. The plenum can be defined at least by the housing and the piston of the syringe. The compressed gas canister can be configured to be moveable relative to the barb. The gas canister can have a ready position in which the gas canister is sealed and is spaced apart from the barb and an engaged position in which the gas canister is in contact with the barb such that the barb pierces a seal of the gas canister. The barb piercing the gas canister seal can allow gas to pressurize the plenum to apply the injection force to the piston, thereby driving the piston relative to the barrel of the syringe to administer the medication.

A power-assisted injection device for administering a liquid medication can include a cartridge, a housing, and an actuator. The cartridge can include a barrel that is configured for holding the liquid medication. The housing can include a body configured for holding the cartridge, first housing helical threads on an interior surface of the body defining a first pitch, second housing helical gear threads on the interior surface of the body defining the second pitch, and a bung configured for linear movement relative to the body and configured for applying an injection force to the cartridge. The actuator can be configured for engaging the first and second housing helical threads and for applying the injection force to the bung.

A power-assisted injection device for administering a liquid medication can include a cartridge and a housing. The cartridge can include a plunger and a barrel that is configured for holding the liquid medication. The housing can include a lower body, an upper body, and a coaxial pinion pair including a first pinion and a second pinion having a common pinion shaft. The housing upper body can be configured for receiving a linear force. The housing upper body can include a rack configured for engaging teeth of the first pinion. The housing lower body can have a rack configured for engaging teeth of the second pinion and configured for applying an injection force to the plunger of the cartridge. A linear force applied to the housing upper body relative to the housing lower body can translate the housing upper body rack to rotate the first and second pinions, and the second pinion transmits movement to the housing lower body rack, thereby translating housing lower body to apply the injection force to the plunger. A pitch of the first pinion and upper body rack can be greater than a pitch of the second pinion and lower body rack such that housing lower body moves less than the housing upper body in response to a unit movement of the housing upper body.

A power-assisted injection device for administering a liquid medication can include a syringe, a housing, and a pulley assembly. The syringe can include a plunger and a barrel that is configured for holding the liquid medication. The housing can include a lower body and an upper body that is moveable relative to the lower body. The pulley assembly can include a pulley wheel and a tether extending about the pulley wheel. The tether can have a first end and a second end with the first end being coupled to the housing upper body and the second end being coupled to the housing lower body. The pulley wheel can be engaged with the syringe plunger. Movement of the housing upper body relative to the housing lower body can create tension in the tether, thereby creating an injection force on the plunger via the pulley wheel.

### BRIEF DESCRIPTION OF THE DRAWINGS

FIG. 1A is view of a power-assisted injection device with dual extension springs shown in a locked, extended position;

FIG. 1B is a view of the embodiment device of FIG. 1A illustrating the springs in a contracted position upon release from the locked position;

FIG. 1C is a view of the embodiment device of FIG. 1A illustrating the needle guard deployed over the needle after actuation of the injection device and delivery of the drug;

FIG. 2A is a schematic view of a second embodiment power-assisted injection device employing a single spring, shown in a locked, extended position;

FIG. 2B is schematic view of the second embodiment device of FIG. 2A, shown in unlocked, extended position;

FIG. 2C is a schematic view of the second embodiment device of FIG. 2B, illustrating the spring in a fully contracted position that is associated with complete administration of the drug;

FIG. 2D is a schematic view of the second embodiment device of FIG. 2C, after activation of the spring-loaded needle guard over the needle;

FIG. 3A is a schematic view of a third embodiment power-assisted injection device employing a double spring in compression, shown in a locked, contracted position;

FIG. 3B is schematic view of the third embodiment device of FIG. 3A, shown in unlocked, contracted position;

FIG. 3C is a schematic view of the third embodiment device of FIG. 3B, illustrating the spring in a fully extended position that is associated with complete administration of the drug;

FIG. 3D is a schematic view of the third embodiment device of FIG. 2C, after activation of the spring-loaded needle guard over the needle;

FIG. 4A is a perspective view of a fourth embodiment injection device before actuation of the plunger;

FIG. 4B is an enlarged, partial cut-say illustrating the gas canister and plunger in the ready position;

FIG. 4C is a schematic view of the fourth embodiment power-assisted injection device employing gas canister illustrated in an intact (unpierced) ready position;

FIG. 4D is a schematic view of the fourth embodiment device of FIG. 4A illustrating the gas canister has been actuated by piercing and a piston in a fully actuated position that is associated with complete administration of the drug;

FIG. 5A is a schematic view of a fifth embodiment power-assisted injection device employing magnets illustrated in a spaced-apart ready position with the plunger fully extended;

FIG. 5B is a schematic view of the fifth embodiment device of FIG. 5A illustrated the attractive force between the magnets and the plunger partially actuated;

FIG. 5C is a schematic view of the fifth embodiment of FIG. 5B illustrating the magnets in contact and the plunger in a fully actuated position that is associated with complete administration of the drug;

FIG. 6A is a partially transparent, perspective view of a sixth embodiment power-assisted injection device including a pair of springs and spring reset device, illustrating a lever of the reset device in the down position and the springs in a pre-loaded ready position;

FIG. 6B is a perspective view of the sixth embodiment device of FIG. 6A, opposite the view of FIG. 6A;

FIG. 6C is a partially transparent, perspective view of the sixth embodiment of FIG. 6A illustrating the lever in an up or retracted position and the device in a ready position;

FIG. 7A is a schematic view of the sixth embodiment device illustrating the ready position shown in FIG. 6C;

FIG. 7B is a schematic view of the sixth embodiment device illustrating the spring in the spring in the fully extended position associated with the complete administration of the drug;

FIG. 7C is a schematic view of the sixth embodiment device illustrating the lever in the in the down position as shown in FIG. 6A;

FIG. 8A is a schematic view of a seventh embodiment power-assisted injection device employing a double spring in compression, shown in a locked, contracted position, including a surface for enabling resetting of the device;

FIG. 8B is a schematic view of the third embodiment device of FIG. 8A, illustrating the spring in a fully extended position that is associated with complete administration of the drug;

FIG. 9A is a perspective, partially cut-away view of an eighth embodiment power-assisted injection device employing a single spring, shown in a locked, extended position in which the spring is in tension and a reset device is in up position;

FIG. 9B is side, partially cut-away view of the eighth embodiment device of FIG. 9A;

FIG. 9C is a side, partially cut-away view of the eighth embodiment device of FIG. 9B, illustrating the spring in a fully contracted position that is associated with complete administration of the drug, with the reset device not yet engaged;

FIG. 9D is a side, partially cut-away view of the eighth embodiment device of FIG. 9C, after activation of the lever of the reset device has moved the spring back into its extended, ready position;

FIG. 9E illustrates a base portion of the eighth embodiment device of FIG. 9A removed from the actuator to illustrate engagement of the reusable base with a syringe;

FIG. 10A is a perspective view of a ninth embodiment power-assisted injection device employing a spiral geared plunger for providing a mechanical advantage to the user, illustrated with the plunger in an extended, ready position;

FIG. 10B is a schematic cross-sectional view of the ninth embodiment device of FIG. 10A;

FIG. 10C is a schematic cross-sectional view of the ninth embodiment device in the ready position shown in FIG. 10A;

FIG. 10D is a schematic cross-sectional view of the ninth embodiment device in the ready position shown in FIG. 10A;

FIG. 10 E is a schematic cross-sectional view of the ninth embodiment device illustrating the plunger in the actuated position associated with complete administration of the drug (not shown);

FIG. 10F is a perspective view of the ninth embodiment device of FIG. 10E;

FIG. 10G is an enlarged cross-sectional view of the device of FIG. 10E;

FIG. 10H is an enlarged cross-sectional view of the device of FIG. 10G illustrating the needle guard deployed over the needle after actuation of the injection device and delivery of the drug;

FIG. 10I is an enlarged, perspective cut-away view of the device illustrated in FIG. 10H;

FIG. 10J is an enlarged, perspective cross-sectional view of the device of FIG. 10H illustrating the needle guard deployed over the needle after actuation of the injection device and delivery of the drug;

FIG. 11A is a perspective view of a tenth embodiment power-assisted injection device employing a dual rack and pinion;

FIG. 11B is a schematic view of the tenth embodiment device of FIG. 11A illustrating the plunger in the extended, ready, unactuated position;

FIG. 11C is a cut-away view of the device of FIG. 11B illustrating the plunger in the extended, ready, unactuated position;

FIG. 11D is a perspective, partially cut-way view of the embodiment of FIG. 11B illustrating the plunger in the extended, ready, unactuated position;

FIG. 11E is a perspective, partially cut-way view of the embodiment device of FIG. 11D illustrating the plunger in a partially the actuated position;

FIG. 11F is a perspective, partially cut-way view of the embodiment device of FIG. 11D illustrating the plunger in the actuated position associated with complete administration of the drug;

FIG. 11G is a schematic view of the embodiment device of FIG. 11A illustrating the plunger in the actuated position associated with complete administration of the drug;

FIG. 11H is a perspective view of the embodiment device of FIG. 11A illustrating the plunger in the actuated position associated with complete administration of the drug;

FIG. 11I is a perspective exploded view of a portion of the ninth embodiment device shown in FIG. 11A illustrating the capability of removing a cap to enable syringe replacement;

FIG. 12A is a schematic view of an eleventh embodiment power-assisted injection device employing a pulley system for a mechanical advantage, illustrating the plunger in an extended, ready position;

FIG. 12B is a schematic view of the embodiment device of FIG. 12A illustrating the plunger in a fully actuated position that is associated with complete administration of the drug;

FIG. 12C illustrates the principle of mechanical advantage employed by the embodiment device of FIG. 12A;

FIG. 13A is a schematic view of a twelfth embodiment power-assisted injection device in an extended, ready position, and employing a pair of springs in series, enabling the force applied to the syringe plunger to vary during actuation;

FIG. 13B is an enlarged schematic view of the injection device of FIG. 13A;

FIG. 13C is a schematic view of the injection device of FIG. 13A illustrating a first spring in an extended position to place the syringe in a partially actuated position;

FIG. 13D is an enlarged schematic view of the injection device of FIG. 13C;

FIG. 13E is a schematic view of the injection device of FIG. 13A illustrating the second spring in an extended position to place syringe in its fully actuated position associated with complete administration of the drug; and

FIG. 13F is an enlarged schematic view of the injection device of FIG. 13E.

### DETAILED DESCRIPTION OF ILLUSTRATIVE EMBODIMENTS

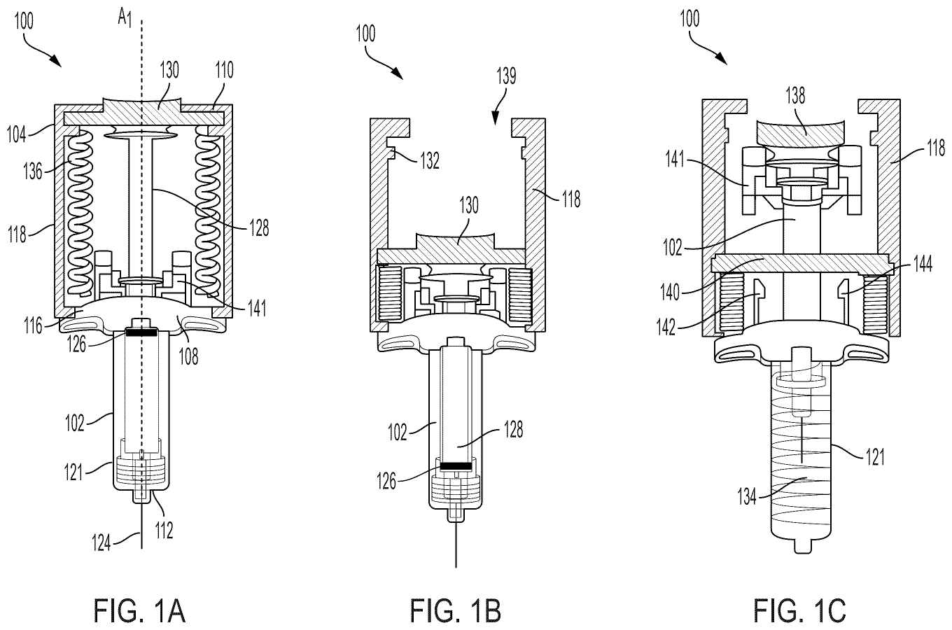

An injection device (e.g., 100, 200, 300, 400, 500, 600, 800, 900, 1000, 1100, 1200, or 1300) can be a power assisted injection device that administers medicament from a syringe. The medicament can be a viscous liquid medication. A viscous liquid medication can have a viscosity of about 10 centipoises to about 375 cp at 20 degrees Celsius. Injection device (e.g., 100, 200, 300, 400, 500, 600, 800, 900, 1000, 1100, 1200, or 1300) can be adapted to receive a syringe 102 (FIG. 1A). Syringe 102 can be adapted such that a user can manually give an injection without injection device (e.g., 100, 200, 300, 400, 500, 600, 800, 900, 1000, 1100, 1200, or 1300). Syringe 102 can be configured to deliver a dose of medicament without the aid of the injection device. Syringe 102 can be coupled to injection device (e.g., 100, 200, 300, 400, 500, 600, 800, 900, 1000, 1100, 1200, or 1300) to reduce user effort to administer an injection. Syringe 102 can include a barrel 122 adapted to hold liquid medication. A flange 103 can extend radially outwardly from the barrel 122. The flange 103 can engage a housing to prevent axial movement of syringe 102 during an injection. The flange 103 can be disposed at a proximal end of the syringe 102.

The medication can be delivered from the barrel 122 to a patient through a discharge port 124. In some examples, the discharge port 124 is a needle. In other examples, the discharge port 124 is a cannula. In other examples, the discharge port 124 is an opening in the barrel such that the injection device (e.g., 100, 200, 300, 400, 500, 600, 800, 900, 1000, 1100, 1200, or 1300) delivers a needle free injection. The discharge port 124 can be in fluid communication with the barrel 122 such that liquid medication can be delivered from the barrel 122 and through the discharge port 124 to the patient. The discharge port 124 can be disposed at a distal end of the syringe 102. A plunger 126 can be moveably received in the barrel 122. Plunger 126 may provide a liquid seal with the barrel 122. Plunger 126 can be moveable relative to barrel 122 such that movement of the plunger 126 toward the discharge port 124 forces medicament out of the discharge port 124.

Injection device (e.g., 100, 200, 300, 400, 500, 600, 800, 900, 1000, 1100, 1200, or 1300) can include a needle guard. A needle guard can be adapted to extend beyond a distal end of a needle after an injection to prevent accidental sticks with the needle. The needle guard can be a passive feature that automatically extends without additional steps from the user. A distal end of a needle guard 121 may include an opening and at least a portion of the discharge port 124 or syringe 102 can extend through the opening when the needle guard 121 is not extended. Needle guard 121 can be fixed relative to housing 104. For example, needle guard 121 can be at least one of rotationally fixed and axially fixed relative to housing 104.

A piston 128 can be operatively associated with the plunger 126. Operatively associated can mean that piston 128 is in contact with plunger 126 during an injection. Movement of the piston 128 can cause movement of the plunger 126 relative to the barrel 122. In some embodiments, the piston 128 and plunger 126 are separate elements. In other embodiments, the piston 128 and plunger 126 are a unitary construct. The piston 128 can be axially spaced from the plunger 126 when an injection device (e.g., 100, 200, 300, 400, 500, 600, 800, 900, 1000, 1100, 1200, or 1300) is in a pre-injection configuration. The piston 128 can move axially into engagement with the plunger 126 as the injection device (e.g., 100, 200, 300, 400, 500, 600, 800, 900, 1000, 1100, 1200, or 1300) transitions from the pre-injection configuration to an injection configuration. In other embodiments, the piston 128 engages the plunger 126 when the injection device (e.g., 100, 200, 300, 400, 500, 600, 800, 900, 1000, 1100, 1200, or 1300) is in the pre-injection configuration. In some embodiments, injection device 100 is reusable by replacing syringe 102 such that piston 128 remains coupled to housing 104 when syringe 102 is replaced. In other embodiments, piston 128 is associated with syringe 102 such that piston 128 is decoupled from housing 104 when syringe 102 is replaced.

Referring now to FIGS. 1A-1C, an injection device 100 is shown. Injection device 100 can be moveable from a pre-injection configuration (FIG. 1A) to an injection configuration (FIG. 1B). Injection device 100 can be movable from an injection configuration to a post-injection configuration (FIG. 1C).

Injection device 100 can include a housing 104. Housing 104 can be manually engageable by a user. Housing 104 can include a proximal end 110 and a distal end 112 opposite the proximal end 110 along a central axis A₁. Housing 104 can include a housing body 118 defined by an outer wall. Housing 104 can be sized and shaped to receive a syringe 102. A needle guard 121 can be coupled to housing body 118. Needle guard 121 can be positionally fixed relative to housing body 118. Needle guard 121 can define a recess to receive at least a portion of the syringe 102. Housing body 118 can define a recess to receive at least a portion of syringe 102 such that syringe 102 is enveloped by the housing 104 and the needle guard 121. Needle guard 121 can be detachably coupled to housing body 118. Syringe 102 can be fixed relative to housing 104 during an injection. Syringe 102 can be moveable relative to housing 104 after medicament is dispensed from syringe 102, as explained in greater detail below.

Injection device 100 can include a flange 130 adapted to move the piston 128 relative to the housing body 118. The flange 130 can be movable relative to the housing body 118 along axis A₁. Flange 130 can engage and move piston 128 relative to housing body 118. In some embodiments, flange 130 and piston 128 are separate elements. In other embodiments, flange 130 and piston 128 are a unitary construct. Flange 130 can be axially spaced from piston 128 when injection device 100 is in the pre-injection configuration. Flange 130 may move into engagement with the piston 128 as the injection device 100 transitions from the pre-injection configuration to the injection configuration. In other embodiments, flange 130 is in contact with piston 128 when injection device 100 is in the pre-injection configuration.

A user may apply a force to flange 130 to move flange 130 relative to housing body 118. A distal end of housing body 118 can include a finger flange 108 such that a user can engage finger flange 108 with two fingers while engaging and applying a force to flange 130 with a thumb. The finger flange 108 can include an opening configured to receive the needle guard 121. Needle guard 121 can be removably received within the opening defined by finger flange 108. The housing body 118 may include an opening 139 such that the user can move flange 130 within the housing body 118.

Housing body 118 can include a latch 132 adapted to define a locked position that inhibits movement of the piston 128 and a released position that enables movement of the piston 128. The latch 132 can be any suitable latch that can define the locked and released positions. In one example, the latch 132 can include be a protrusion that engages an underside of flange 130 when the latch 132 is in the locked position, thereby retaining flange 130 in the locked position until release. At least one of latch 132 and flange 130 can be flexible such that one of flange 130 and latch 132 flexes out of engagement with the other of flange 130 and latch 132 when a sufficient force is applied. A user may apply an initiation force to the flange 130 to move the latch 132 from the locked position to the released position. The initiation force may can be less than the injection force applied by the motive force device 136. The initiation force can be about 15 Newtons to about 40 Newtons.

Injection device 100 can include a motive force device 136 adapted for applying an injection force to the plunger 126 to administer the medication while the latch 132 is in the released position. The injection force can be separate and distinct from the initiation force. A user can apply the initiation force and the motive force device 136 can apply the injection force. The initiation force can be applied over an initiation time period. The injection force can be applied over an injection time period. The initiation time period can be about 1 second. The injection time period can be about 1 second to about 10 seconds, about 10 seconds to about 30 seconds, about 30 seconds to about 45 seconds, about 45 seconds to about 1 minute, or at least about 1 minute. The initiation force can be applied to the flange 130. The injection force can be applied to the plunger 126. In some embodiments, the motive force device 136 applies a sufficient injection force to plunger 126 to complete the injection even if the user does not manually apply any force to the flange 130 after the latch 132 is in the released position. The injection device 100 can be adapted for administering the liquid medication upon release of the latch 132 from the locked position at least in part via the motive force device 136 applying the injection force to the plunger 126 of the syringe 102. The motive force device 136 may apply the injection force to the plunger 126 via the piston 128. The injection force can be about 10 Newtons to about 25 Newtons, about 25 Newtons to about 50 Newtons, about 50 Newtons to about 75 Newtons, or about 75 Newtons to about 100 Newtons. In some examples, the injection device 100 includes a 27-gauge needle to deliver a liquid having a viscosity of about 1 centipoise and the injection force is about 14 Newtons for a 3 second injection. In other examples, the injection device 100 includes a 27-gauge needle to deliver a liquid having a viscosity of about 60 centipoise and the injection force is about 50 Newtons for a 10 second injection.

In some embodiments, the motive force device 136 applies the injection force without requiring additional force from the user. In other embodiments, the injection force of the motive force device 136 is insufficient to move the plunger 126 while the latch 132 is released without an external force applied by a user. Thus, the user must apply a force to flange 130 in addition to the force from motive force device 136 to perform the injection. The motive force device 136 can be coupled to the flange 130. Alternatively, the motive force device 136 can be coupled to the piston 128.

The motive force device 136 can transition from an expanded state to a relaxed state as the injection device 100 moves from the pre-firing configuration to the firing configuration. The motive force device 136 can be a biasing element. The motive force device 136 can be at least one spring engaged with the housing body 118. The at least one spring can be a pair of springs engaged with the housing body 118. The housing body 118 can include a base 116 proximate an upper portion of the barrel 122. The at least one spring of the motive force device 136 can be in tension between the base 116 and the flange 130. Alternatively, the motive force device 136 could be a gas fired piston.

The injection device can include a syringe holder 141 (labeled in FIG. 1C). The syringe holder 141 can couple to the barrel 122 of the syringe 102. The barrel 122 can be positionally fixed relative to the syringe holder 141 in at least an axial direction when coupled thereto. The needle guard 121 can be configured to move axially relative to the syringe holder 141 to transition between an initial position (FIGS. 1A and 1B), wherein the distal end of the discharge port 124 extends out of the needle guard 121, and a retracted position (FIG. 1C), wherein the distal end of the discharge port 124 is retracted into the needle guard 121. In some examples, the syringe holder 141 and needle guard 121 are part of the UltraSafe Plus Injection System, marketed by Becton, Dickinson and Company.

A return force device 134 can be adapted to move the syringe 102 from the injection position (FIG. 1B) to the post-injection position (FIG. 1C). The return force device 134 can be a biasing element (e.g., a spring). The return force device 134 can exert a force against the needle guard 121 and the syringe holder 141 in opposing directions to move syringe holder 141 (and hence the syringe 102) proximally relative to housing 104 (including the needle guard 121). The discharge port 124 can be within the needle guard 121 when the syringe 102 is in the post-injection position.

The flange 130 may include a first portion 138 and a second portion 140 (FIG. 1C). First portion 138 can be detachably coupled to second portion 140. Motive force device 136 can be coupled to second portion 140 such that motive force device 136 moves first portion 138 and second portion 140 from the pre-injection position to the injection position. A latch (not shown) can couple first portion 138 to second portion 140. The first portion 138 and second portion 140 may move together from the pre-injection position to the injection position.

A disengagement member 142 can be coupled to the housing body 118 and adapted to disengage the first portion 138 from the second portion 140. The disengagement member 142 can include a protrusion 144 adapted to engage the syringe holder 141 to retain the needle guard 121 in an initial position (FIGS. 1A and 1B). At least one of the first portion 138 and the second portion 140 can cause the disengagement member 142 to disengage from the syringe holder 141 such that the return force device 134 can move the syringe 102 from the initial position to a retracted position (FIG. 1C).

At least a portion of the injection device 100 can be reusable. For example, a user can move the second portion 140 of flange 130 to engage the latch 132. The user can may replace syringe 102 with a new syringe and reuse the injection device 100. The syringe 102 can be detached from the injection device 100 by decoupling the syringe barrel 122 from the syringe holder 141. A new syringe 102 can be coupled to the injection device 100 such that the injection device 100 can be used again.

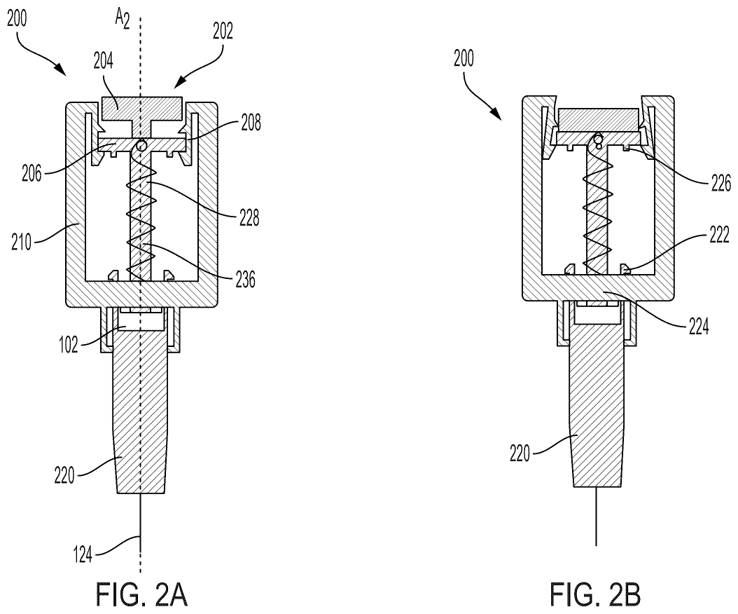

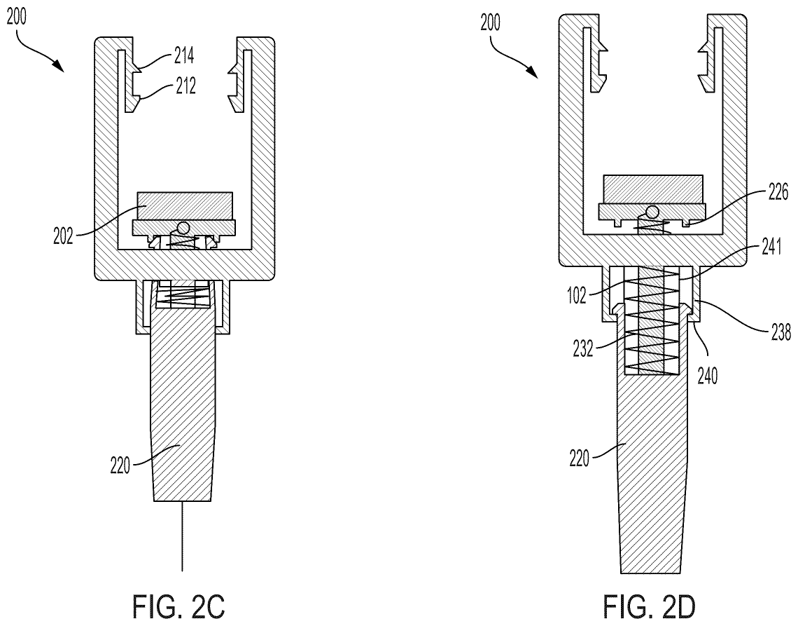

FIGS. 2A-2D illustrate another embodiment of an injection device 200. Injection device 200 can include a housing 210 adapted to receive syringe 102. Injection device 200 can include a plunger 228 adapted to apply the injection force syringe 102. A motive force device 236 can be coupled to plunger 228 to move plunger 228 relative to housing 210. Alternatively, motive force device 236 can be coupled to a flange 202 coupled to plunger 228. The motive force device 236 can be a spring that is coaxial with plunger 228. The spring can be in tension between a base of housing 210 and the flange 202. Motive force device 236 can define a recess with at least a portion of plunger 228 within the recess.

Flange 202 can include a first portion 204 and a second portion 206. First portion 204 can be moveable relative to second portion 206. Second portion 206 can be fixed to plunger 228. Second portion 206 and plunger 228 can be a unitary construct. Movement of first portion 204 relative to second portion 206 can initiate an injection, as explained below. A portion of first portion 204 can be nested within second portion. First portion 204 can include a first portion central axis extending along a maximum length of first portion 204 and second portion 206 can include a second portion central axis extending along a maximum length of second portion 206, be coaxial with second portion 206. First portion central axis can be coaxial with second portion central axis.

Housing 210 can include a latch 208 adapted to define a locked position that inhibits movement of plunger 228 and a released position that enables movement of the plunger 228. Latch 208 can be engageable with flange 202. For example, latch 208 can be engaged with second portion 206. In some embodiments, latch 208 can be a flexible arm that moves from a locked position (FIG. 2A) to a released position (FIG. 2B). Flange 202 can be moveable relative to housing 210 when latch 208 is in the released position.

Latch 208 can include a protrusion 212 (FIG. 2C) engageable with an underside of flange 202 to arrest axial movement of flange 202 in the distal direction when latch 208 is in the locked position. Latch 208 can retain flange 202 in the locked position until release. Latch 208 can include a ramped surface 214 engageable with the first portion 204. First portion 204 can be moveable relative to the second portion 206 while the second portion 206 is engaged with the protrusion 212. For example, first portion 204 can be translatable along axis A₂ (FIG. 2A) relative to second portion 206. Movement of first portion 204 relative to second portion 206 can move latch 208 from the locked position to the released position. In some examples, a release force can be required to move the latch 208 from the locked position to the released position. The release force can be about 15 N to about 40 N. First portion 204 can engage ramped surface 214 such that protrusion 212 moves out of engagement with the second portion 206. In some embodiments, latch 208 includes a cantilevered beam arm that flexes radially outwardly away from the central axis of the housing 210 when the first portion 204 engages the ramped surface 214. Latch 208 can move into contact with an inner surface of housing 210 as latch 208 flexes from the locked position to the released position.

Latch 208 can be a sidewall that continuously extends circumferentially around an opening sized to receive flange 202. Alternatively, latch 208 can be two or more sidewalls that are spaced from each other around a perimeter defining an opening to receive flange 202.

A first end of motive force device 236 can be coupled to flange 202. A second end of motive force device 236 can be coupled to housing 210. For example, motive force device 236 can be coupled to the second portion 206 of flange 202 and a distal portion of housing 210. Motive force device 236 can be in tension between flange 202 and distal portion of housing 210.

Injection device (e.g., 200, 300, 400, 500, 600, 800, 900, 1100, or 1200) can include a needle guard 220 movable relative to a housing such that a needle is covered after an injection. Needle guard 220 can be moveable relative to at least one of housing 210 and syringe 102 from an injection position to a post-injection position. Needle guard 220 can envelope at least a portion of syringe 102 in the post-injection position. Needle guard 220 can extend distally beyond a distal end of discharge port 124 when needle guard is in the post-injection position. Discharge port 124 can extend distally from a distal end of the needle guard 220 when the needle guard 220 is in the injection position.

Needle guard 220 can be at least temporarily fixed relative to housing 210 before an injection. Needle guard 220 can include an arm 222 engaged with a lower wall 224 of housing 210. Lower wall 224 can include an opening such that arm 222 extends from a first side of lower wall 224 to a second side of lower wall 224 that is opposite the first side along axis A₂. Arm 222 can include a detent engageable with the lower wall 224 to at least temporarily maintain the position of the needle guard 220 with respect to the housing 210. Arm 222 can be moveable from an engaged position to a disengaged position. Arm 222 can be a cantilevered beam extending proximally from a proximal end of a body of needle guard 220. The detent of arm 222 can be engaged with the lower wall 224 in the engaged position and disengaged from the lower wall 224 in the disengaged position. Arm 222 can be flexible from the engaged position to the disengaged position. Needle guard 220 can be movable relative to housing 210 when arm 222 is in the disengaged position.

Flange 202 can be adapted to move the arm 222 from the engaged position to the disengaged position. Flange 202 can include a disengagement member 226 that moves arm 222 as flange 202 moves axially toward the lower wall 224. For example, one of disengagement member 226 and arm 222 can include a ramped surface engageable with the other of disengagement member 226 and arm 222 such that axial translation of flange 202 moves arm 222 radially into the disengaged position. Needle guard 220 can move from the injection position toward the post-injection position when the arm 222 is in the disengaged position.

A lower housing 238 can be coupled to housing 210. Lower housing 238 can include a sidewall sized and shaped to moveably receive needle guard 220. Lower housing 238 can include a ledge 240 adapted to engage arm 222 when needle guard 220 is in the post-injection position. The injection device 200 can include a syringe holder 241 (labeled in FIG. 2D). The syringe holder 241 can couple to the barrel 122 of the syringe 102. The barrel 122 can be positionally fixed relative to the syringe holder 241 in at least an axial direction when coupled thereto. The syringe holder 241 can be positionally fixed relative to the housing 210. The needle guard 220 can be configured to move axially relative to the syringe holder 241 to transition between the injection position (FIGS. 2A, 2B, and 2C), wherein the distal end of the discharge port 124 extends out of the needle guard 220, and a post-injection position (FIG. 2D), wherein the distal end of the discharge port 124 is retracted into the needle guard 220. In some examples, the syringe holder 241 and needle guard 220 part of the UltraSafe Plus Injection System, marketed by Becton, Dickinson and Company.

A biasing element 232 can provide force to move needle guard 220 from the injection position to the post-injection position. Biasing element 232 can include a first portion exerting a force against a lower surface of lower wall 224 or against the syringe holder 241. Biasing element 232 can include a second portion exerting a force against needle guard 220. Biasing element 232 can be adapted to expand when arm 222 is in the disengaged position. For example, biasing element 232 can be a spring that is compressed when the needle guard 220 is in the injection position such that the needle guard 220 expands when no longer constrained by the arm 222.

The injection device 200 can be reusable. A user may disengage syringe holder 241 and needle guard 220 from lower housing 238 and remove the syringe 102. For example, a user may apply a force radially inwardly to disengage arm 222 from lower housing 238 and pull needle guard 220 away from lower housing 238. A user can then move plunger 228 proximally until second portion 206 engages latch 208. A user may then insert a new syringe 102 into the needle guard 220. Needle guard 220 can then be recoupled to housing 210 and moved proximally until arms 222 engage housing 210.

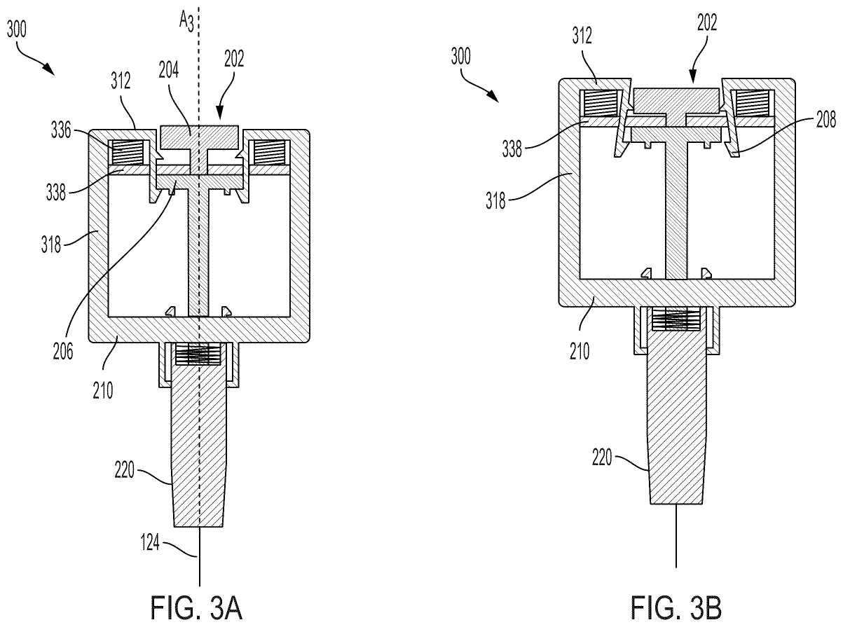

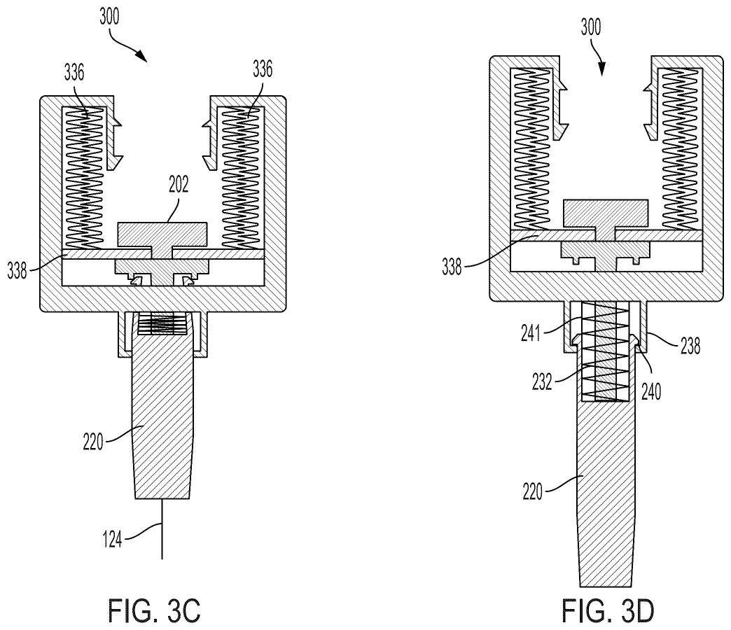

FIGS. 3A-3D illustrate another embodiment of an injection device 300. Injection device 300 can be similar to injection device 200. However, a motive force device 336 of injection device 300 can be different from motive force device 236 of injection device 200. For example, motive force device 336 can include at least one spring in compression between a housing base 312 and a housing flange 338 while latch 208 is in the locked position. The motive force device 336 can be adapted to move the flange 202 relative to housing 210 when the latch 208 is in the released position.

Housing 310 can include a base 312 proximate an upper portion of a housing body 318. Housing flange 338 can be a bar, plate, or rod adapted to contact and move flange 202 for an injection. Housing flange 338 can have a width similar to the inner width of the housing 310 to maintain alignment of the housing flange 338 relative to housing 310 as the housing flange 338 moves to the post-injection position. Maintaining alignment of the housing flange 338 can help ensure a smooth and complete delivery of the dose of medicament. Housing flange 338 can include a central axis extending along a maximum length of housing flange 338. Central axis of housing flange can be perpendicular to a central axis A₃ of injection device 300.

Movement of the first portion 204 of flange 202 relative to second portion 206 can initiate the injection by moving the latch 208 to the released position as previously discussed. The motive force device 336 can exert a force against housing flange 338. Housing flange 338 can apply the injection force to flange 202 in response to force applied by motive force device 336 such that flange 202 moves relative to housing 310. In some examples, the injection device 300 is configured to couple to an existing injection system. One injection system contemplated for use with the injection device 300 is the UltraSafe Plus Injection System, marketed by Becton, Dickinson and Company.

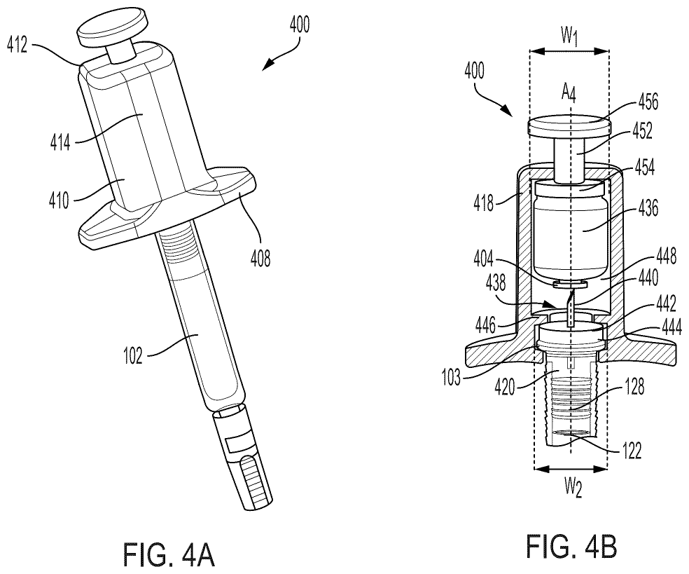

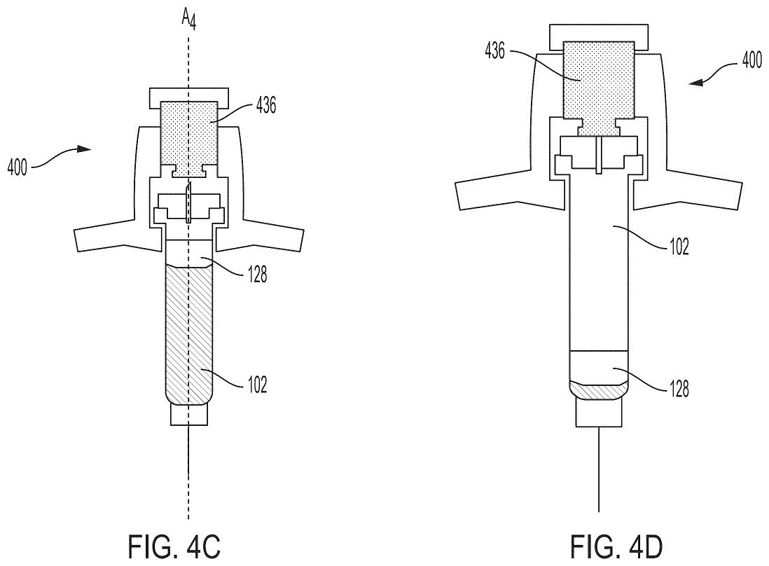

FIGS. 4A-4D illustrate another embodiment of an injection device 400. Injection device 400 can include a housing 410 with a housing body 418 adapted for holding the syringe 102. Housing 410 can include a power source to move syringe plunger 126 relative to the barrel 122. The power source can be a compressed gas canister 436. Gas within the canister can be hexafluoroacetone (HFA), liquified petroleum gas, helium, nitrogen, oxygen, or VapourSoft manufactured by Recipharm AB having a headquarters in Stockholm, Sweden. Activating the canister 436 can release the compressed gas to move the syringe plunger 126.

The canister 436 can include a seal 404 that can be pierced or moved to allow the compressed gas to exit the canister. The seal 404 can be positioned at a distal end of the canister 436. In some embodiments, the seal 404 can be a pierceable or frangible end portion of the canister 436. In other embodiments, the seal 404 can be a plug engaged with an opening sidewall of the canister 436, thereby sealing the canister.

A barb assembly 438 can be adapted to pierce or move the seal 404 to allow gas to exit the canister 436. The barb assembly 438 can include a barb 440 adapted to pierce the seal 404. The barb 440 can have a pointed end that facilitates piercing the seal 404. Alternatively, the barb 440 can have a blunt or rounded end that pushes the seal 404 to disengage from the opening sidewall of the canister 436. The barb 440 can include an opening extending therethrough to allow gas from the canister 436 to flow through the barb 440.

The barb assembly 438 can include a support structure 442 adapted to support the barb 440. The support structure 442 can fix the position of the barb assembly relative to the canister 436 during an injection. The support structure 442 can be operatively associated with the flange 103 of the syringe 102 to axially fix the position of the barb 440 relative to the syringe 102 during an injection. The barb 440 can have a height as measured along central axis A₄ that is greater than a height of the support structure 442 such that gas from the canister 436 can flow through the barb 440 from a first side of the support structure 442 to a second side opposite the first side. In some embodiments, the support structure 442 can sealingly engage the flange 103 of the syringe to reduce or prevent gas from escaping at the interface of the flange 103 and support structure 442.

The housing 410 can define a channel 444 to receive the syringe flange 103 and support structure 442. The housing 410 can include a partition 446 that separates the channel 444 from an aperture 448. The partition 446 can be a protrusion that extends inward from the housing body 418. The partition 446 can include an opening such that at least one of the barb 440 and a portion of the canister 436 extends through the opening. The support structure 442 can be slightly compressed between the flange 103 and the partition 446 to prevent unintentional damage to the flange 103 or firing of the injection device 400 before intended use. In other embodiments, the partition 446 can be positioned between the support structure 442 and the flange 103. In still other embodiments, housing 410 does not include partition 446 such that canister 436 and barb assembly 438 are disposed in aperture 448. The injection device 400 can include a plenum 420 between the piston 128 and the flange 103 of the syringe 102. The gas from the canister 436 can fill the plenum 420 when the injection device 400 is activated. At least a portion of support structure 442 can be positioned in plenum 420.

In some embodiments, the support structure 442 can be fixed relative to the housing 410. In other embodiments, the support structure 442 is moveable relative to the housing 410. For example, support structure 442 can be coupled to canister 436 such that canister moves support structure 442 as canister 436 moves relative to housing 410. Partition 446 can stop axial movement of support structure 442 when support structure is movable relative to housing 410. Canister 436 can continue moving axially when the support structure 442 stops moving such that there is relative movement between the canister 436 and support structure 442. Barb 440 can be fixed to support structure 442 such that barb 440 is fixed relative to canister 436 when support structure 442 is fixed relative to canister 436 and barb 440 is moveable relative to canister 436 when support structure 442 is movable relative to canister 436.

The canister 432 can be in slidable contact with the housing body 418 that defines the aperture 448. The aperture 448 can have a width Wi in a plane that is perpendicular to the central axis A₄. The width W₁ can be greater than a width W₂ of the channel 444. There can be an interference fit between canister 436 and sidewall of aperture 448 to prevent unintentional movement of the canister 436 prior to use.

The canister 436 can be moveable relative to housing 410 between a ready position (FIG. 4C) and an engaged position (FIG. 4D). The canister 436 can be spaced (e.g., axially spaced along axis A₄) from the barb 440 in the ready position. The canister 436 can be sealed in the ready position. The canister 436 can move relative to the barb 440 as the canister 436 moves from the ready position to the engaged position. The canister 436 can slide in the aperture 448 upon movement from the ready position to the engaged position. The barb 440 can pierce the seal of the canister 436 as the canister 436 moves to the engaged position. Gas can exit the canister 436 as barb 440 pierces the seal to pressurize the plenum and apply an injection force to the piston 128, thereby driving the piston 128 relative to the barrel of the syringe to administer the medication.

Canister 436 can include an activator 452 (FIG. 4B) engageable by a user to move the canister 436 from the ready position to the engaged position. Activator 452 can include a first end 454 operatively associated with canister 436. In some embodiments, first end 454 is coupled to canister 436 when canister 436 is in the ready position. In other embodiments, first end 454 is axially spaced from canister 436 along central axis A₄ when canister 436 is in the ready position. First end 454 can move into contact with the canister 436 as the activator 452 is moved by the user relative to housing 410. The activator can be disposed in an opening of a proximal portion of housing 410.

A distal end of housing body 418 can include a finger flange 408 engageable by a user during use of injection device 400. For example, a user may engage finger flange 408 with two fingers while engaging activator 452 with a thumb. User may apply a force to activator 452 to move canister 436 relative to housing 410. In some examples, the injection device 400 is configured to couple to an existing injection system. One injection system contemplated for use with the injection device 400 is the UltraSafe Plus Injection System, marketed by Becton, Dickinson and Company.

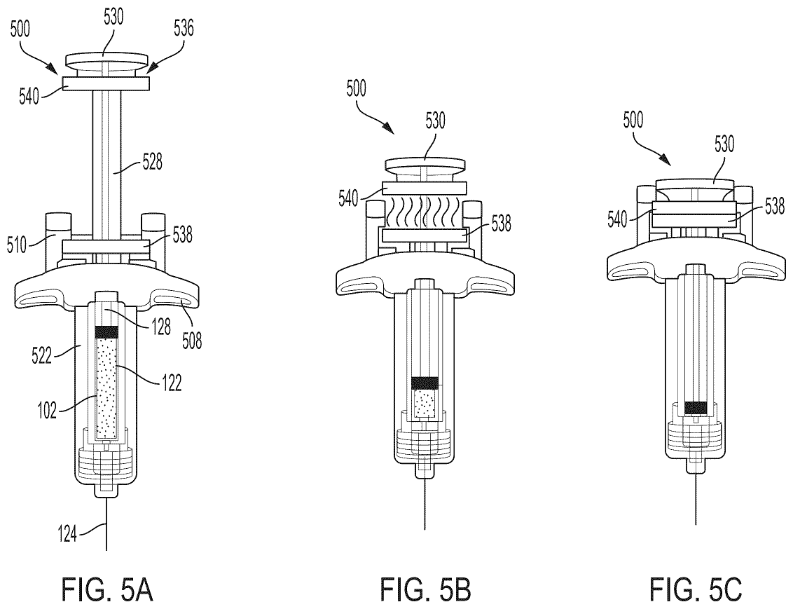

FIGS. 5A to 5C illustrate another embodiment of an injection device 500. Injection device 500 can include a housing 510 to receive at least a portion of syringe 102. The housing 510 can be a syringe holder. A needle guard 522 can be coupled to the housing 510. At least one of the housing 510 and the needle guard 522 can couple to the barrel 122 of the syringe 102. The barrel 122 can be positionally fixed relative to the housing 510 in at least an axial direction when coupled thereto. The needle guard 522 can be configured to move axially relative to the housing 510 to transition between an injection position, wherein the distal end of the discharge port 124 extends out of the needle guard 522 (FIGS. 5B and 5C), and a post-injection position (not shown), wherein the distal end of the discharge port 124 is retracted into the needle guard 522. In some examples, the housing 510 and needle guard 522 are part of the UltraSafe Plus Injection System, marketed by Becton, Dickinson and Company.

Injection device 500 can include a plunger 528 operatively associated with piston 128 of syringe 102 to dispense medicament from syringe 102. For example, plunger 528 can move piston 128 relative to barrel 122 of syringe 102 to dispense medicament from the discharge port 124.

Injection device 500 can include a motive force device 536 adapted for applying an injection force to plunger 528 to administer medication. Motive force device 536 can include a pair of magnetic components. First magnetic component 538 and second magnetic component 540 each be a magnet oriented to attract the other of the first magnetic component 538 and the second magnetic component 540. Alternatively, one of first magnetic component 538 and second magnetic component 540 can be a magnet and the other of first magnetic component 538 and second magnetic component 540 can be a ferrous structure to attract the magnet. First magnetic component 538 can be coupled to housing 510 proximate barrel 122 of syringe 102. Second magnetic component 540 can be coupled to plunger 528 at or near a plunger flange 530.

A distal end of housing body 518 can include a flange 508 engageable by a user to activate injection device 500. For example, a user can engage flange 508 with two fingers while engaging piston flange 530 with a thumb. A user can initiate an injection by applying a force to piston flange 530 to move plunger 528 relative to housing 510. The force applied by the motive force device 536 can increase as the distance between the first magnetic component 538 and second magnetic component 540 decreases. The motive force device 536 can provide a force of about 14 N to about 100 N. In some embodiments, motive force device 536 exerts at least some force before plunger 528 begins to move. In other embodiments, motive force device 536 applies an injection force only when plunger 528 has moved a selected distance. For example, the selected distance can be about 10%, about 20%, about 30%, about 40%, or about 50% of the travel distance of plunger 528. First magnetic component 538 and second magnetic component 540 can be spaced from each other until the completion of the administration of the medication. In some embodiments, first magnetic component 538 contacts second magnetic component 540 when injection device 500 is in a post injection configuration. In other embodiments, first magnetic component 538 is spaced from second magnetic component 540 when injection device 500 is in the post injection configuration.

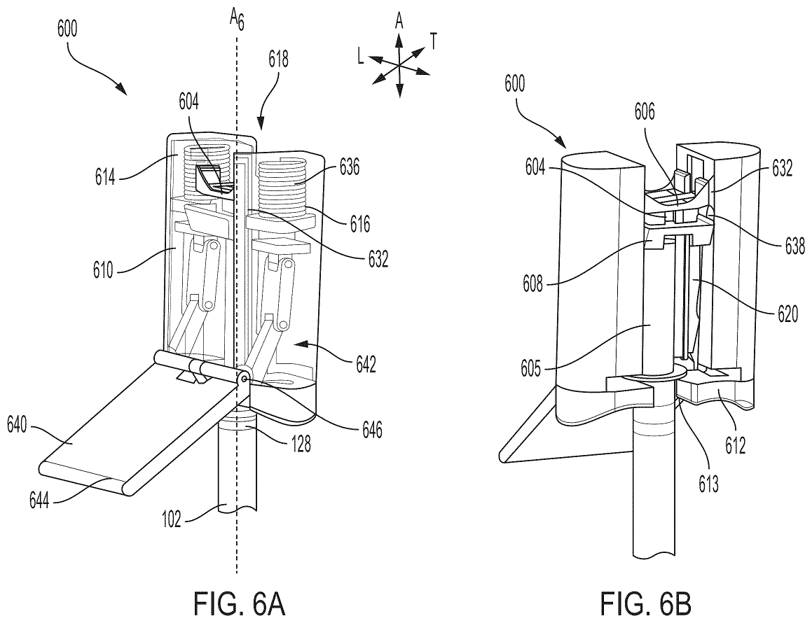

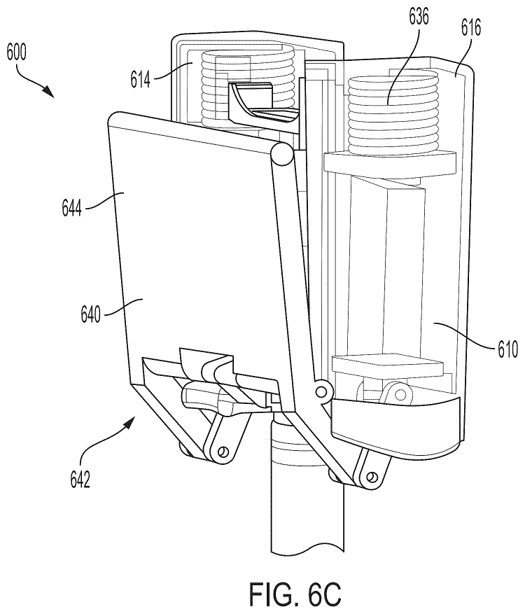

FIGS. 6A-6C illustrate another embodiment of an injection device 600 which can include a force device to facilitate an injection and a load actuator adapted to load the force device. Injection device 600 can include a housing 610 adapted for holding syringe 102. Housing 610 can include a housing body 618, a lower wall 612, and an upper wall 622. Lower wall 612 can include a channel 613 to receive a portion of syringe 102. Channel 613 can extend through a surface of lower wall 612 such that syringe 102 can be loaded into the channel in a transverse direction T that is perpendicular to each of a lateral direction L and an axial direction A which can be parallel with a central axis A₆. Channel 613 can extend from a first side of lower wall 612 to a second side of lower wall 612 opposite the first side in the transverse direction T. Channel 613 can extend through lower wall 612 such that syringe 102 can be loaded from the first side or the second side of lower wall along transverse direction T. Lower wall 612 can include a groove to secure syringe 102 in position.

Injection device can include a plunger 605 adapted to apply an injection force to piston 128 to dispense medicament from syringe 102. A flange 604 can be coupled to plunger 605. Flange 604 can be operatively associated with plunger 605 such that flange 604 contacts plunger 605 to move plunger 605 relative to housing body 618. For example, flange 604 can move plunger 605 along axis A₆ relative to housing body 618. Flange 604 can include a first portion 606 and a second portion 608. First portion 606 can be movable relative to second portion 608. For example, first portion 606 can be translatable along axis A₆ relative to second portion 608.

Housing body 618, lower wall 612, and upper wall 622 can define a recess to receive the force device. Housing 610 can be adapted to receive a biasing element 636. Biasing element 636 can be at least one spring. The biasing element 636 can provide an injection force of about 14 N to about 100 N. Biasing element 636 can be adapted to apply an injection force to piston 128. For example, biasing element 636 can apply the injection force to flange 604, thereby applying the injection force to piston 128. Housing body 618 can include a first receiving area 614 and a second receiving area 616 adapted to receive a first biasing element and a second biasing element, respectively. First receiving area 614 can be spaced from second receiving area in the lateral direction L. Flange 604 can extend at least partially into a channel 620 in each of first receiving area 614 and second receiving area 616. Alternatively, housing body 618 could be a single element that receives each of first biasing element and second biasing element or a single biasing element.

Second portion 608 of flange 604 can extend into the channels 620 such that biasing element 636 is in contact with second portion 608 during an injection. Biasing element 636 can be compressed between second portion 608 and housing 610. For example, first biasing element can be compressed between second portion 608 and upper wall 622 of first receiving area 614 and second biasing element can be compressed between second portion and upper wall 622 of second receiving area 616 when the biasing element 636 is in a ready position.

Housing body 618 can include a latch 632 adapted to define a locked position that inhibits movement of the plunger 605 and a released position that enables movement of the plunger 605. Latch 632 can be a protrusion that engages an underside of second portion 608 of flange 604 when latch 632 is in the locked position, thereby retaining flange 604 in the locked position until release. At least one of latch 632 and first portion 606 of flange 604 can be flexible to allow first portion 606 to translate along axis A₆ from a first side of latch 632 to a second side of latch 632 opposite the first side. For example, latch 632 can be a flexible arm cantilevered from at least one of first receiving area 614 and second receiving area 616. Latch 632 can include a protrusion 638 engageable by first portion 606. First portion 606 can contact protrusion 638 and move latch 632 out of engagement with second portion 608 as first portion 606 moves relative to second portion 608 thereby moving latch 632 from the locked position to the released position. Biasing element 636 can move plunger 605 relative to housing 610 when latch 632 is in the released position. Second portion 608 of flange 604 may slide within channel 620 from a ready position to a fired position as the plunger 605 moves relative to housing 610.

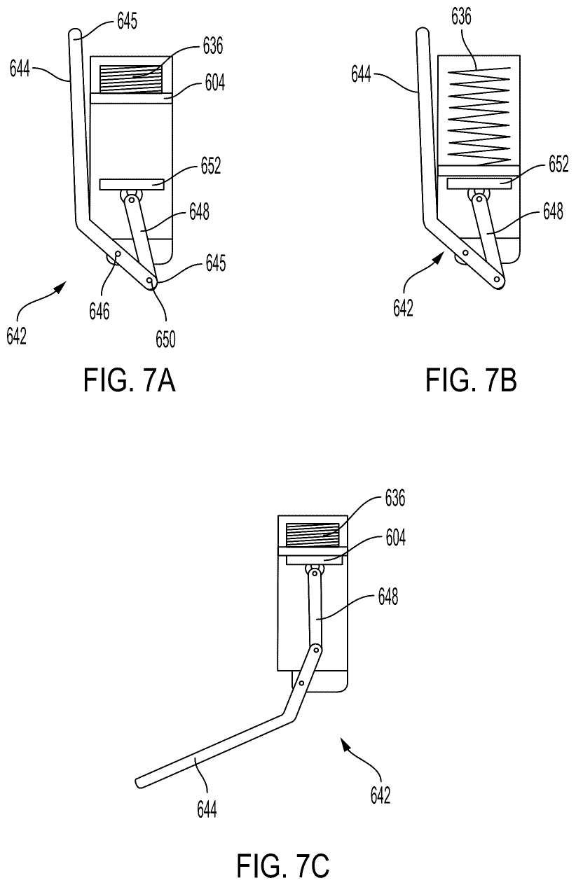

Referring to FIGS. 6A-6C and 7A-7C, injection device 600 can include a load actuator 640 adapted for loading the biasing element 636. Injection device 600 can be adapted for administering the liquid medication upon loading of the biasing element 636. Load actuator 640 can include a linkage assembly 642 adapted to move the at least one biasing element 636 from a fired position (FIG. 7B) to a ready position (FIGS. 6A and 7C). Linkage assembly 642 can include a lever arm 644 coupled to housing body 618. Lever arm 644 can be pivotable about a pivot 646 fixed to housing body 618. Lever arm 644 can be configured to rotate about pivot 646. Pivot 646 can be fixed to an external surface of housing body 618. Alternatively, pivot 646 can be fixed to lower wall 612 of housing 610. In another alternative, pivot 646 can be coupled within at least one of first receiving area 614 and second receiving area 616. Lever arm 644 can include a first end 643 and a second end 645 opposite the first end 643 and a length as measured from the first end to the second end. The length can be the longest dimension of lever arm 644. Pivot 646 can be positioned between first end 643 and second end 645.

Linkage assembly 642 can include at least one linkage arm 648 pivotably coupled to lever arm 644. Linkage arm 648 can be pivotably coupled to an end of lever arm 644 such that applying a force to lever arm 644 transmits a loading force through linkage arm 648 upward (i.e., in axial direction A) to compress the biasing element 636. Linkage arm 658 can be pivotably coupled to lever arm 644 at connection point 650. Connection point 650 can be positioned between second end 645 and pivot 646. Pivot 646 can be positioned between first end 643 and connection point 650. Second end 645 can be positioned within housing body 618. Although only one linkage arm is shown, it is understood that any number of desired linkage arms can be coupled together between lever arm and platform 652.

Linkage assembly 642 can include a platform 652 adapted to engage and move flange 604 as the biasing element 636 is being compressed. For example, platform 652 can move flange 604 along axis A₆. Platform 652 can move flange 604 along axis A₆ in response to rotational movement of lever arm 644. Although only one linkage arm is shown, it is understood that any number of desired linkage arms can be coupled together between lever arm 644 and platform 652. Lever arm 644 can be in a first position (FIG. 7A) when injection device 600 is in a ready position. Platform 652 can be in a lowest position when lever arm 644 is in the first position. A space between platform 652 and flange 604 can be at a maximum distance along axis A₆ when lever arm 644 is in the first position. Flange 604 can move toward platform 652 as biasing element 636 expands and injection device 600 dispenses a dose of medication. In some embodiments, flange 604 moves into contact with platform 652 as biasing element 636 expands. In other embodiments, flange 604 remains spaced from platform 652 even when biasing element 636 is fully expanded within housing 610. Lever arm 644 can be rotated about pivot 646 from first position to a second position (FIG. 7C). Platform 652 can contact flange 604 as lever arm 644 is pivoted from the first position to the second position. Biasing element 636 can be in its most compressed state when lever arm 644 is in second position. Lever arm 644 can then be rotated back to the first position (FIG. 7A) such that platform 652 moves away from flange 604 and injection device is ready to dispense a dose of medication.

Linkage assembly 642 can include a pair of linkage arms 648 each positioned within one of the first receiving area 614 and second receiving area 616. In some embodiments, biasing element 636 is a pair of springs engaged with opposing ends of flange 604 and applying a force to lever arm 644 transmits a loading force through the linkage arms 648 upward to compress the springs.

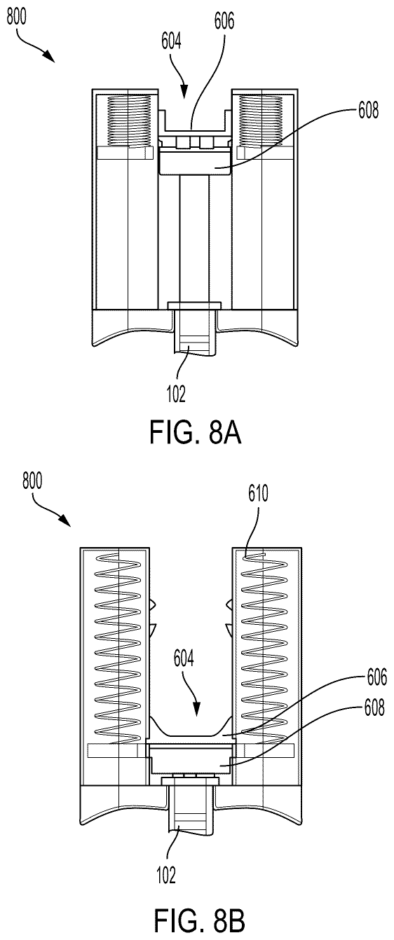

FIGS. 8A-8B illustrate another embodiment of an injection device 800. Injection device 800 can be similar to injection device 600 but injection device 800 does not include a linkage assembly. Instead, flange 604 can be manually moved relative to housing 610 from a first position (FIG. 8A) to a second position (FIG. 8B). For example, a user can apply a force to second portion 608 to move flange 604 relative to housing 610. Alternatively, injection device 800 can be a single use device such that flange 604 cannot be returned to the first position.

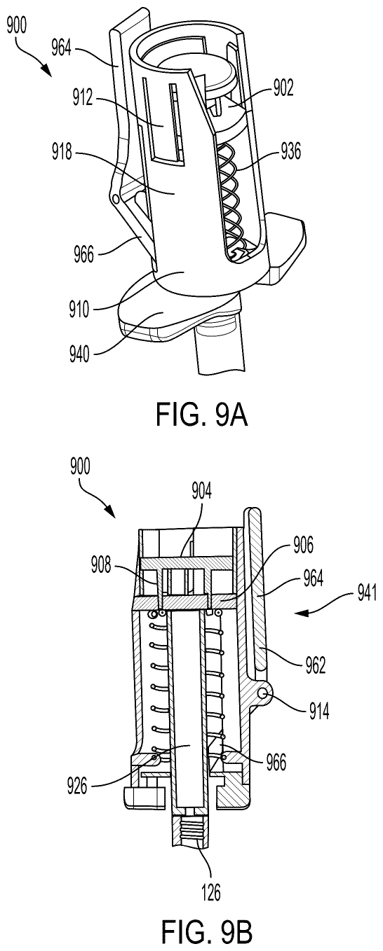

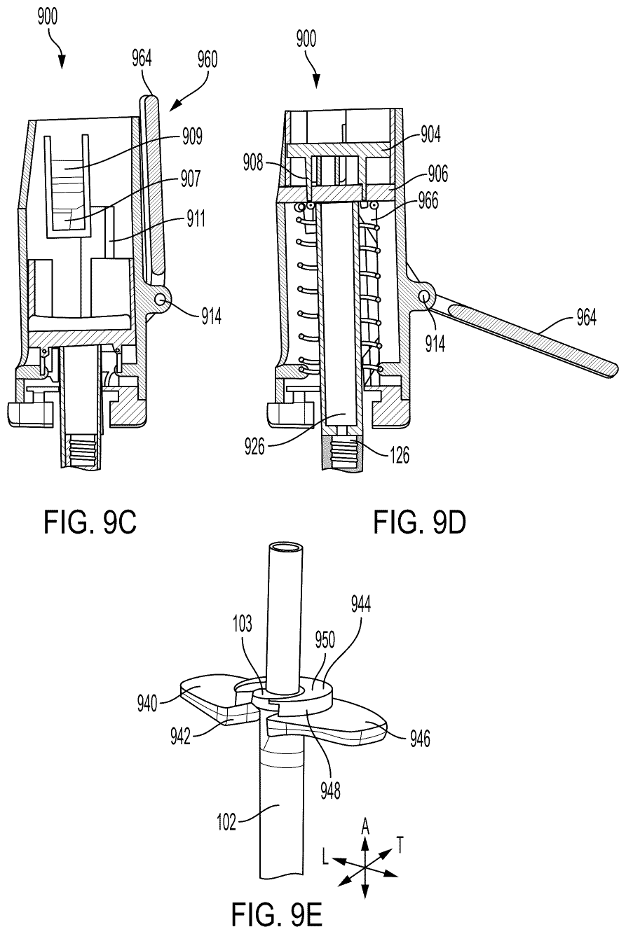

FIGS. 9A-9E illustrate another embodiment of an injection device 900. Injection device 900 can be a power-assisted injection device for administering a liquid medication. Injection device 900 can include a housing 910 with a housing body 918 to receive at least a portion of syringe 102. Injection device 900 can include a plunger 926 operatively associated with piston 128 of syringe 102 to dispense medicament from syringe 102. For example, plunger 926 can move piston 128 relative to barrel 122 of syringe 102 to dispense medicament from discharge port 124.

A base flange 940 can be coupled to a distal portion of housing 910. In some embodiments, base flange 940 is removably coupled to housing 910. In other embodiments, base flange 940 is fixed to housing 910. Base flange 940 can be adapted for engaging the syringe 102. Base flange 940 can include a channel 942 such that at least a portion of syringe 102 can be loaded into the channel 942 in a transverse direction T that is perpendicular to each of a lateral direction L and axial direction A. In some embodiments, syringe 102 can be coupled to base flange 940 before base flange 940 is secured to housing 910. A rim 944 can be coupled to a body 946 of base flange 940. Rim 944 can protrude from a proximal surface of base flange 940. Rim 944 can include a sidewall 948 that extends around at least a portion of channel 942. Rim 944 can include a top wall 950 such that top wall 950, sidewall 948, and body 946 define a receiving area for syringe flange 103. Top wall 950 can extend from a surface of sidewall 948 which faces channel 942. Body 946 can be positioned on a first side of syringe flange 103 and top wall 950 can be positioned on a second side of syringe flange 103 opposite the first side along axial direction A. Rim 944 can be sized and dimensioned to fit with housing body 918. A distal end of housing body 918 can engage a proximal surface of body 946. Base flange 940 can be coupled to housing body 918 by threaded engagement, snap fit, adhesive, or welding.

Injection device 900 can include a biasing element 936 to move plunger 926 relative to housing 910 to eject medicament out of syringe 102. Biasing element 936 can be a spring. Biasing element 936 can be a spring that is coaxially disposed about the plunger 926. A flange 902 can be coupled to plunger 926. Flange 902 can extend radially outwardly beyond an outer perimeter of plunger 926. Biasing element 936 can be coupled to flange 902 and a distal end of housing 910. Biasing element 936 can be a spring in tension such that the spring pulls flange 902 toward distal end of housing 910.

Flange 902 can be similar to flange 202 in that flange 902 includes a first portion 904 and a second portion 906 similar to first portion 204 and second portion 206. First portion 904 can include one or more legs 908 that extend through an aperture in second portion 906. First portion 904 can be at least partially nested within second portion 906. First portion 904 can be movable relative to second portion 906. For example, first portion 904 can be movable in an axial direction A relative to second portion 906.

Housing 910 can include a latch 912 moveable between a locked configuration wherein movement of flange 902 is prevented and an unlocked configuration that allows movement of flange 902. Latch 912 can be a cantilevered beam within an opening in housing body 918. Latch 912 can include protrusions that extend into a central opening of housing body 918. A first protrusion 907 (FIG. 9C) can engage and prevent movement of second portion 906 relative to housing 910 (e.g., axial movement) of flange 902 when latch 912 is in the locked configuration. First portion 904 can engage a second protrusion 909 as first portion 904 moves relative to second portion 906 to transition latch 912 from the locked configuration to the unlocked configuration. Latch 912 can flex outwardly such that first protrusion disengages from second portion 906, thereby allowing plunger 926 to move relative to housing body 918. First protrusion 907 and second protrusion 909 can each be positioned within a recess defined by housing body 918.

Injection device 900 can include a load actuator 941 adapted for loading biasing element 936. Injection device 900 can be adapted for administering the liquid medication upon loading of the biasing element 936. Load actuator 941 can be similar to load actuator 640. Load actuator 941 can include a linkage assembly 962 adapted to move the at least one biasing element 936 from a fired configuration (FIG. 9C) to a ready configuration (FIG. 9B). Linkage assembly 962 can be adapted to transmit an axial force to biasing element 936 in response to rotational movement of linkage assembly 962.

Linkage assembly 962 can include at least one linkage arm 966 coupled to lever arm 964. Linkage arm 966 can be coupled to an end of lever arm 964 such that applying a force to lever arm 964 transmits a loading force through linkage arm 966 (i.e., in axial direction A) to compress biasing element 936. Linkage arm 966 can be fixed to lever arm 964. Linkage arm 966 can be rotationally fixed to lever arm 964. Linkage arm 966 and lever arm 964 can be a monolithic element. Linkage arm 966 can extend through an opening 911 in housing body 918 such that lever arm 964 can be engaged by a user to move the biasing element 936 within housing 910. Although only one linkage arm is shown, it is understood that linkage assembly 962 can include any number of desired linkage arms.

Linkage arm 966 can include a first end and a second end opposite the first end along a central axis of linkage arm 966. First end of linkage arm 966 can be coupled to lever arm 964. Second end of linkage arm 966 can be pivotably coupled to flange 902. Second end of linkage arm 966 can be detachably coupled to flange 902. Lever arm 964 can include a first end and a second end opposite the first end along a central axis of lever am. The linkage arm central axis can be transverse to the lever arm central axis.

In some embodiments, second end of linkage arm 966 is coupled to flange 902. In other embodiments, a platform (not shown) can be coupled to linkage assembly 962 to move flange 902 along axial direction A. The platform can be similar to platform 652. Flange 604 can move along axial direction A in response to rotational movement of lever arm 964. Lever arm 644 can pivot about a pivot 914. Lever arm 644 can be fixed to linkage arm 966 such that lever arm 964 and linkage arm 966 both pivot about pivot 914. Pivot 914 can be an axle. Pivot 914 can be fixed to housing 910. Pivot 914 can be located on a first side of housing 910 and channel 942 can extend to through an outer surface of base flange 940 on a second side of housing 910 opposite the first side. Flange 902 can move from a ready position to a fired position along axial direction A. The ready position can be proximal to the fired position. Pivot 914 can be axially positioned between the ready position and the fired position. Pivot 914 can be positioned on an outer surface of housing 910 and be positioned between the ready position and the fired position of the flange 902 along an axis generally parallel to a central axis of housing 910.

Lever arm 964 can be in a first position (FIGS. 9A and 9B) when injection device 900 is in the ready configuration. The second end of lever arm 964 can be in a lowest position when lever arm 964 is in the first position. A space between the second end of lever arm 964 and flange 902 can be at a maximum distance along axial direction A when lever arm 964 is in the first position. Flange 602 can move toward the second end of lever arm 964 as biasing element 936 expands and injection device 900 dispenses a dose of medication. In some embodiments, flange 902 moves into contact with the second end of lever arm 964 as biasing element 936 expands. In other embodiments, flange 902 remains spaced from second end of lever arm 964 when lever arm 964 is in the first position and biasing element 936 is fully expanded within housing 9010.

Lever arm 964 can be rotated from first position to a second position (FIG. 9D). Lever arm 964 can contact flange 902 as lever arm 964 is pivoted from the first position to the second position. Lever arm 964 can move flange 902 as lever arm 964 is pivoted from the first position to the second position. Biasing element 936 can be in its most compressed state when lever arm 964 is in second position. Lever arm 964 can then be rotated back to the first position such that injection device is ready to dispense a dose of medication.

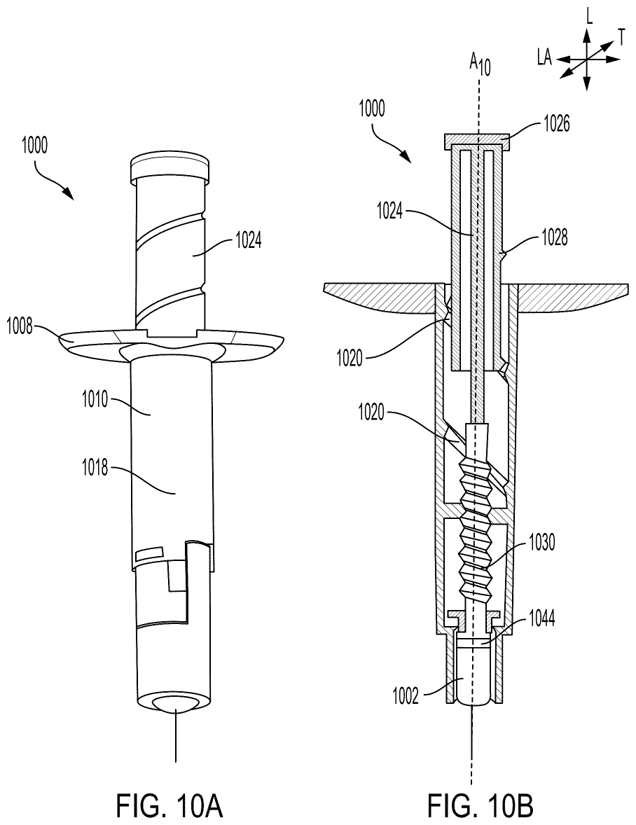

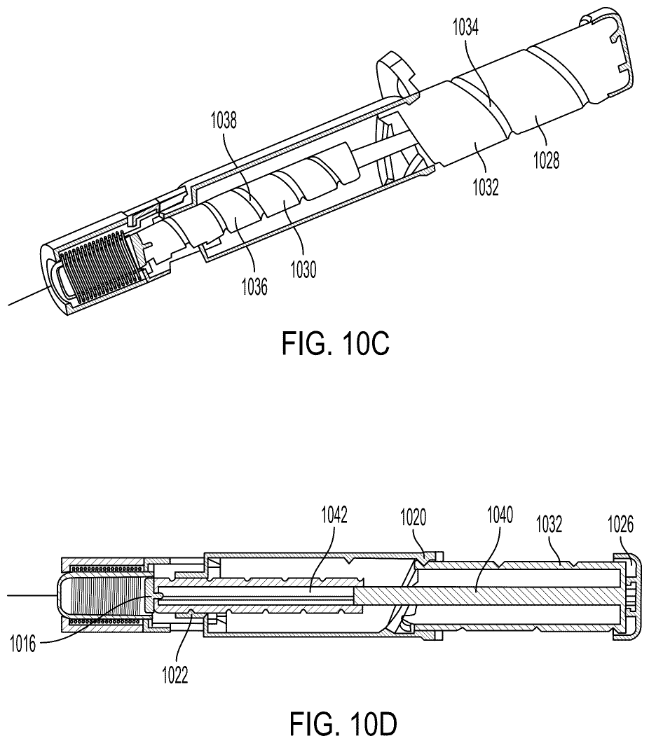

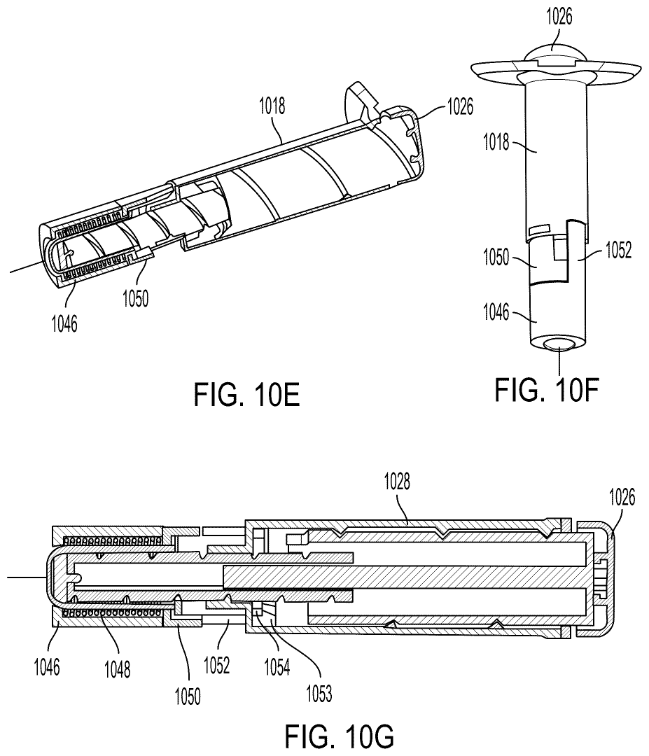

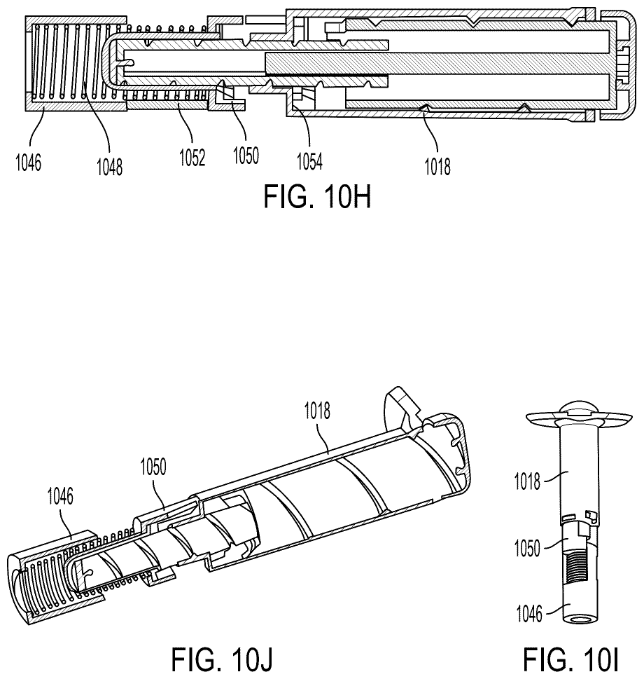

FIGS. 10A-10J illustrate another embodiment of an injection device 1000. Injection device 1000 can be a power assisted injection device for administering a liquid medication. Injection device 1000 can be adapted to provide a mechanical advantage such that an injection force applied to medicament in a cartridge is greater than a force applied by a user. Injection device 1000 can include a cartridge 1002 with a barrel adapted for holding the liquid medication. Cartridge 1002 can include a needle in fluid communication with the barrel such that medicament is dispensed from the barrel through the needle and to an injection site. Cartridge 1002 can be syringe 102.

Injection device 1000 can include a housing 1010 with a housing body 1018 adapted for holding cartridge 1002. Housing body 1018 can include a finger flange 1008 similar to flange 130. Housing body 1018 can include an internal surface and an external surface opposite the internal surface. Housing body 1018 can define an internal recess. Housing body 1018 can include a first end and a second end spaced from the first end along a central axis A₁₀. Central axis A₁₀ can extend in a longitudinal direction. A lateral direction LA can be perpendicular to longitudinal direction. A transverse direction T can be perpendicular to each of lateral direction and longitudinal direction L.

Housing body 1018 can include a first thread 1020 configured to engage an actuator 1024. First thread 1020 can be on the internal surface of housing body 1018. First thread 1020 can be a helical thread. First thread 1020 can be positioned in a proximal portion of housing body 1018. In some embodiments, first thread 1020 continuously extends circumferentially around inner surface of housing body 1018 through at least one revolution. In other embodiments, first thread 1020 extends less than one revolution.

Housing body 1018 can include a second thread 1022 (FIG. 10D). Second thread 1022 can be configured to engage an actuator 1024. Second thread 1022 can be a helical thread. First thread 1020 can have a first thread pitch and second thread 1022 can have a second thread pitch. First thread pitch can be different from second thread pitch. First thread pitch can be different from second thread pitch to yield a mechanical advantage relative to a force applied by the user to the actuator 1024. Second thread 1022 can be positioned in a distal portion of housing body 1018. In some embodiments, second thread 1022 continuously extends circumferentially around inner surface of housing body 1018 through at least one revolution. In other embodiments, second thread 1022 extends less than one revolution.

Actuator 1024 can be movable relative to housing body 1018. Actuator 1024 can be longitudinally movable relative to housing body 1018. Actuator 1024 can rotate about axis A₁₀ as actuator 1024 moves longitudinally via the threaded engagement between first thread 1020 and actuator 1024. In some embodiments, actuator 1024 can be engaged with first thread 1020 and second thread 1022 simultaneously. In other embodiments, actuator 1024 engages first thread 1020 and second thread 1022 sequentially.

Actuator 1024 can include a first member 1028 and a second member 1030. First member 1028 can be movable relative to second member 1030. First member 1028 can include recess configured to receive at least a portion of second member 1030. Second member 1030 can be moveable relative to first member 1028 along the longitudinal axis L.

Second member 1030 can move toward a distal end of the injection device 1000 during an injection sequence.

In some examples, first member 1028 can include a first member thread 1034. First member thread 1034 can be an outer thread. In other examples, an outer surface of first member 1028 can include a worm gear. First member 1028 can include a first member body 1032 with first member thread 1034 configured for engaging first thread 1020. First member thread 1034 can be positioned on an exterior surface of first member body 1032. First member thread 1034 can be recessed into the exterior surface of first member body 1032. First thread 1020 of housing body 1018 can protrude laterally from the interior surface of housing body 1018 toward central axis A₁₀. Outer thread 1034 can be a helical thread.

Second member 1030 can include a second member body 1036 with a second member thread 1038 configured for engaging second thread 1022. Second member thread 1038 can be positioned on an exterior surface of second member body 1036. In some embodiments, second member thread 1038 extends along the longitudinal length of second member body 1036. In other embodiments, second member thread 1038 extends less than the full length of second member body 1036. Second member 1030 can have a cylindrical shape. Second member 1030 and first member 1028 can be mutually rotationally constrained relative to housing body 1018. In some embodiments, exterior surface of second member body 1036 can be radially spaced from inner surface of first member body 1032. In other embodiments, exterior surface of second member body 1036 contacts interior surface of first member body 1032.

A shaft 1040 can be fixed to first member body 1032. Shaft 1040 can be longitudinally coaxial with first member body 1032. Shaft 1040 can extend distally from a proximal portion of first member body 1032. Second member 1030 can include a recess 1042 adapted for receiving shaft 1040. Rotation of one of the first member 1028 and second member 1030 can transmit torque to the other of the first member 1028 and the second member 1030. For example, shaft 1040 and recess 1042 can have a non-circular cross-sectional shape taken along a plane perpendicular to axis A₁ such that rotation of one of first member 1028 and second member 1030 transmits torque to the other of first member 1028 and second member 1030. Linear movement of first member 1028 can cause first member 1028 to rotate via the first threads 1020. Rotation of the first member 1028 can transmit rotation to second member 1030 via shaft 1040 and recess 1042. Rotation of second member 1030 can cause linear movement of second member 1030 relative to housing body 1018. Second member thread 1038 can have a thread pitch that is different from a pitch of first member thread 1034. A pitch of first member thread 1034 can be greater than a pitch of second member thread 1038 such that second member 1030 axial translation is less than first member 1028 translation. Second member 1030 and first member 1028 can rotate at the same rotational rate while first member 1028 axially translates faster than second member 1030.

Actuator 1024 can rotate as a user applies a linear force to actuator 1024. A user can apply a liner force to a cap 1026 coupled to actuator 1024. Cap 1026 can be rotatably coupled to actuator 1024. Cap 1026 can remain rotationally fixed relative to housing body 1018 as actuator 1024 translates axially relative to housing body 1018. Cap 1026 can remain rotationally fixed relative to housing body 1018 as actuator 1024 rotates relative to housing body 1018.

Injection device 1000 can include a bung 1044 adapted for applying an injection force to cartridge 1002. Bung 1044 can be coupled to actuator 1024. Bung 1044 can be coupled to second member 1030. Bung 1044 can be coupled to a distal end of second member 1030. Bung 1044 can form a fluid seal with cartridge 1002. In other embodiments, bung 1044 can push piston 128 of syringe 102. Bung 1044 can apply an injection force to dispense medicament from cartridge 1002 as actuator 1024 moves relative to housing body 1018. In some embodiments, bung 1044 is rotatably coupled to second member 1030 such that bung 1044 is rotationally fixed relative to cartridge 1002 as second member 1030 rotates. In other embodiments, bung 1044 is rotationally fixed to second member 1030. Second member 1030 can have a maximum length in the lateral direction or transverse direction such that at least a portion of second member 1030 can enter barrel of cartridge 1002 to dispense medicament (FIG. 10H). In other embodiments, bung 1044 includes a stem coupled to second member 1030 having a longitudinal length sufficient to dispense the dose of medicament from cartridge 1002 without second member 1030 entering barrel of cartridge 1002.

Injection device 1000 can include a needle guard 1046. Needle guard 1046 can extend distally beyond a distal end of needle to prevent accidental sticks from needle. Needle guard 1046 can be fixed relative to housing body 1018 prior to an injection. Needle guard 1046 can be fixed relative to housing body 1018 after an injection. Needle guard 1046 can move relative to housing body 1018 after medicament has been dispensed from cartridge 1002. A needle guard biasing element 1048 can apply a force to needle guard 1046 to move needle guard 1046 relative to housing body 1018.

A collar 1050 can be adapted to fix cartridge 1002 relative to housing body 1018. Collar 1050 can be positioned between housing body 1018 and needle guard 1046. Collar 1050 can include a central opening with cartridge 3 1002 disposed in central opening. Needle guard 1046 can be at least temporarily engaged with one of collar 1050 and housing body 1018. Needle guard 1046 can include a needle guard arm 1052 that engages a rim 1054 of housing body 1018. For example, needle guard arm 1052 can include a protrusion 1053 (FIG. 10G) that engages rim 1054 when needle guard 1046 is in the retracted position. At least one of housing body 1018 and needle guard 1046 can include a lock. Needle guard 1046 can engage collar 1050 (FIG. 10H) to lock needle guard in the extended position.

Actuator 1024 can disengage needle guard arm 1052 from rim 1054. One of first member 1028 and arm 1052 can include a ramped surface such that axial movement of first member 1028 relative to needle guard arm 1052 causes radial movement of needle guard arm 1052. Needle guard arm 1052 can disengage from rim 1054 as needle guard arm 1052 moves radially such that needle guard 1046 can move relative to housing body 1018 to an extended position. Needle guard biasing element 1048 can maintain needle guard in the extended position.

Needle guard 1046 can extend distally beyond a distal end of needle when needle guard 1046 is in the extended position. A portion of actuator 1024 can be observable from an exterior of injection device 1000 when needle guard 1046 is in the extended position. For example, a portion of second member 1030 can be observable through an exterior of injection device 1000 when needle guard 1046 is in the extended position after an injection. This can provide visual indication to a user that the injection is complete and the injection device 1000 is in a locked configuration.

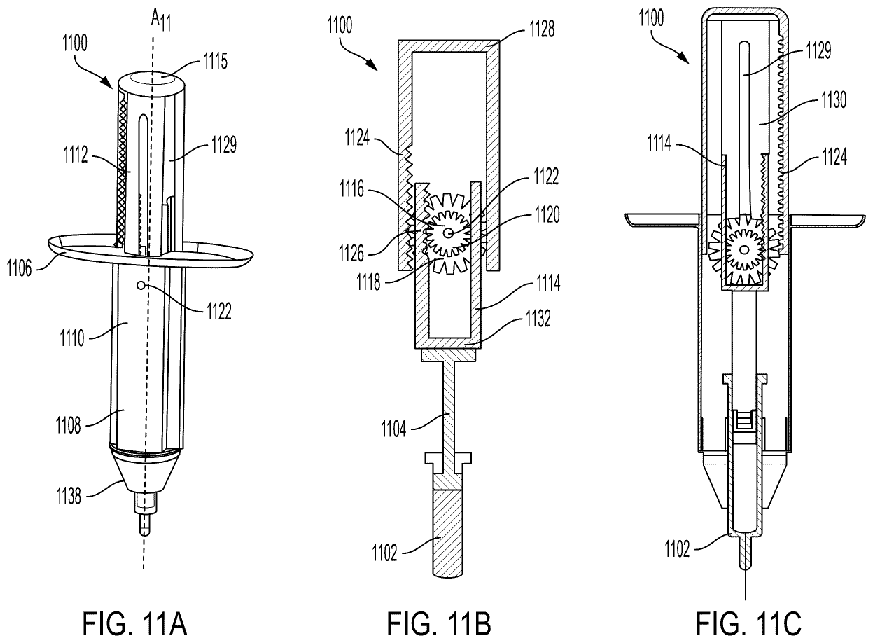

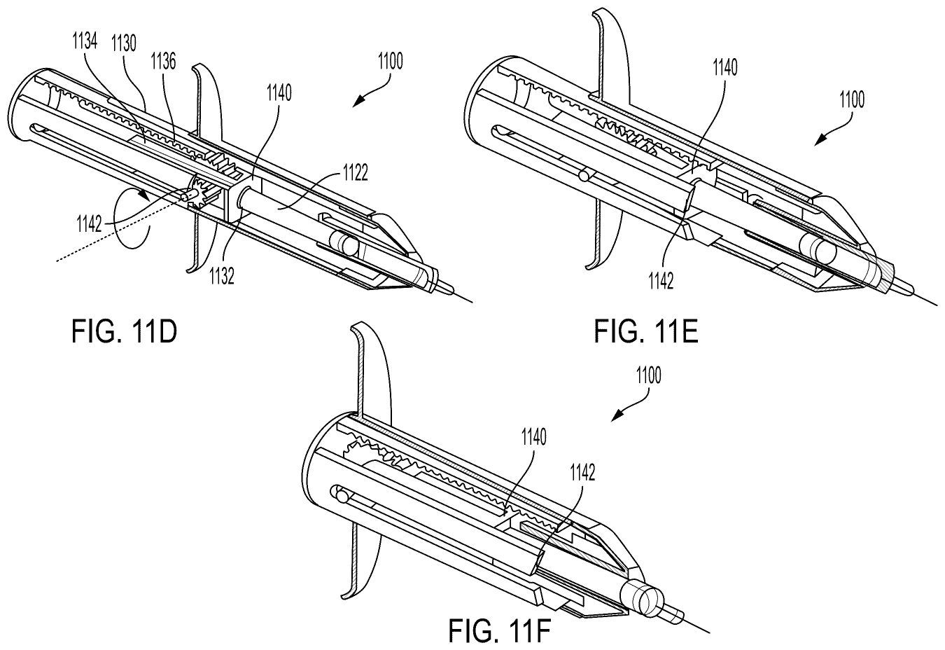

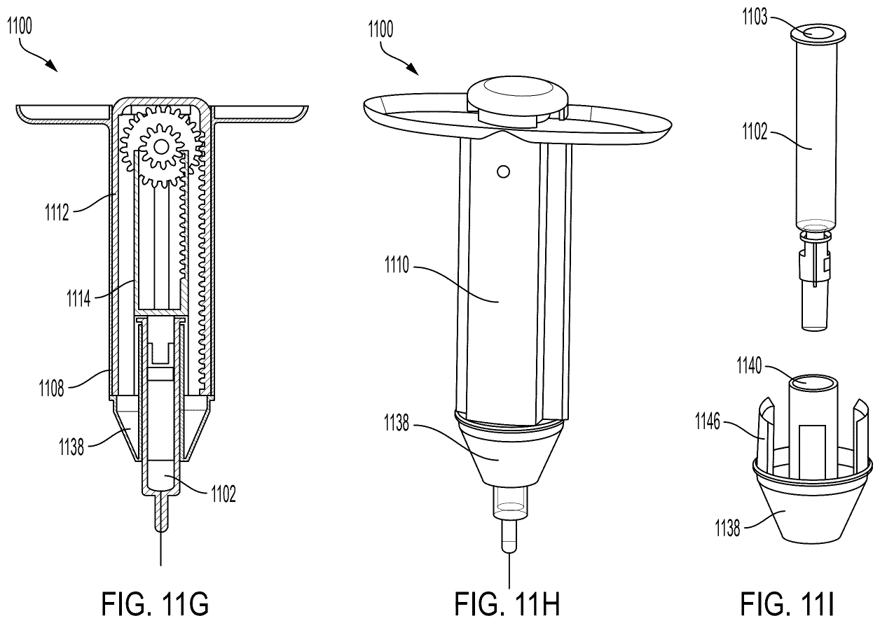

FIGS. 11A-11I illustrate another embodiment of an injection device 1100. Injection device 1100 can be a power assisted injection device for administering a liquid medication. Injection device 1100 can be adapted to provide a mechanical advantage such that an injection force applied to medicament in a cartridge is greater than a force applied by a user. Injection device 1100 can be adapted to receive a cartridge 1102 with a barrel adapted for holding the liquid medication. Cartridge 1102 can include a plunger 1104 and a needle in fluid communication with the barrel such that movement of plunger 1104 relative to cartridge 1102 dispenses medicament from the barrel through the needle and to an injection site. Cartridge 1102 can be syringe 102. Cartridge 1102 can be fixed relative to housing 1110. Cartridge 1102 can be longitudinally fixed relative to housing 1110.

Injection device 1100 can include a housing 1110 adapted for holding cartridge 1102. Housing 1110 can include a first end and a second end spaced from the first end along a central axis A₁₁. Central axis A₁₁ can extend in a longitudinal direction. A lateral direction LA can be perpendicular to longitudinal direction L. A transverse direction T can be perpendicular to each of lateral direction and longitudinal direction L.

Housing 1110 can include an upper body 1112 and a lower body 1114. Upper body 1112 and lower body can be movable relative to each other. Housing 1110 can include a shell 1108. Upper body 1112 can be movable relative to shell 1108. Lower body 1114 can be movable relative to shell 1108. Upper body 1112 and lower body 1114 can be movable relative to shell 1108. Upper body 1112 and lower body 1114 can be movable in longitudinal direction L relative to shell 1108. A finger flange 1106 can extend from shell 1108.

One of upper body 1112 and lower body 1114 can be telescopically nested within the other of upper body 1112 and lower body 1114. A pinion assembly 1116 can be coupled to at least one of upper body 1112 and lower body 1114. Pinion assembly 1116 can include a pinion pair coupled to a shaft 1122. Pinion pair can include a first pinion gear 1118 and a second pinion gear 1120. First pinion gear 1118 and second pinion gear 1120 can be coaxial. First pinion gear 1118 and second pinion gear 1120 can each be adapted to rotate about a central axis of shaft 1122. First pinion gear 1118 and second pinion gear 1120 can be coupled to the same shaft 1122. Pinion assembly 1116 can be axially fixed in the longitudinal direction relative to housing 1110. Shaft 1122 can be rotatable relative to housing 1110. Shaft 1122 can be axially fixed relative to housing 1110. Shaft 1122 can be coupled to shell 1108.

Upper body 1112 can be adapted to engage first pinion gear 1118. Upper body 1112 can include an upper rack 1124 adapted for engaging teeth of first pinion gear 1118. Upper rack 1124 and first pinion gear 1118 can have a first gear pitch.

Lower body 1114 can be adapted to engage second pinion gear 1120. Lower body 1114 can include a lower rack 1126 adapted for engaging teeth of second pinion gear 1120. Lower rack 1126 and second pinion gear 1120 can have a second gear pitch. First gear pitch can be different from second gear pitch. First gear pitch can be greater than second gear pitch to provide a mechanical advantage when moving lower body 1114 relative to housing 1110. In some embodiments, lower body 1114 moves 0.5 millimeters in response to 1 millimeter of movement of upper body 1112 relative to housing 1110.

An injection force can be applied to upper body 1112. For example, a user can manually apply an axial force to a proximal end 1115 of upper body 1112 with a thumb or finger. Upper body 1112 can include an end wall 1128 adapted to be engaged by a user to apply an axial force to upper body 1112. One or more beams 1130 can extend from end wall 1128. Beam 1130 can be a cantilevered beam. Beam 1130 can extend along a central axis in the longitudinal direction L from a first end to a second end opposite the first end. First end of beam 1130 can be coupled to end wall 1128. Second end of beam 1130 can be a free end. A space 1129 can separate beams 1130 from each other. Beams 1130 can be circumferentially spaced from each other. At least one beam 1130 can include upper rack 1124. Upper rack 1124 can be formed on a single beam or on opposing beams such that upper rack 1124 engages opposing ends of first pinion gear 1118. Shaft 1122 can be positioned in at least one of the spaces 1129 between beams 1130.

Housing 1110 can include a guide to at least partially maintain the alignment of beams 1130 relative to each other. In some embodiments, the guide can be a protrusion that extends from an interior surface of shell 1108 into the space 1129 between beams. In other embodiments, the guide is a recess in the sidewall of housing 1110 that receives beam 1130.

Lower body 1114 can be adapted to apply an injection force to plunger 1104. Lower body 1114 can include an end wall 1132 operatively associated with plunger 1104 such that the lower body 1114 applies an injection force to plunger 1104. For example, plunger 1104 can be coupled to end wall 1132. In some embodiments, plunger 1104 and lower body 1114 are a unitary construct. In other embodiments, plunger 1104 and lower body 1114 are separate elements that are operatively associated with each other. One or more lower body beams 1134 can extend from end wall 1132. Lower body beam 1134 can be a cantilevered beam. Lower body beam 1134 can include a first end and a second end spaced from the first end along a lower body beam central axis. First end of lower body beam 1134 can be coupled to lower body end wall 1132. Second end of lower body beam 1134 can be a free end. A space 1136 can separate lower body beams 1134 from each other. Lower body beams 1134 can be spaced from each other about a perimeter of lower body 1114. A halo can couple second end of lower body beams 1134 to each other.

At least one lower body beam 1134 can include lower rack 1126. Shaft 1122 can be positioned in at least one of the spaces 1136 between lower body beam 1134. First pinion gear 1118 can be position in at least one of the spaces 1136. An outer surface of lower body 1114 can be laterally spaced from an inner surface of upper body 1112.

A distal end 1140 of lower body beams 1134 can be positioned distally beyond a distal end 1142 of upper body beams 1130 when injection device 1100 is in a ready configuration (FIG. 11D). Distal end 1140 of lower body beams 1134 can be aligned along longitudinal axis L with distal end 1142 of upper body beams 1130 at an instance during an injection (FIG. 11E). Distal end 1140 of lower body beams 1134 can be positioned proximally of distal end 1142 of upper body beams 1130 when injection device 1100 is in a fired configuration (FIG. 11F). At least one of upper body 1112 and shell 1108 can include a lock out feature. The lock out feature can be a projection on one of the upper body 1112 and shell 1108 received within a recess on the other of upper body 1112 and shell 1108 to lock the injection device in the fired configuration.

A tip 1138 can be engaged with housing 1110. Tip 1138 can be removably coupled to housing 1110. Tip 1138 can be coupled to a distal end of housing 1110. Tip 1138 can be coupled to shell 1108 of housing 1110. Tip 1138 can be configured to engage cartridge 1102. Tip 1138 can include an extension 1144 adapted to receive cartridge 1102. An end surface of extension 1144 can be adapted to engage a flange 1103 on cartridge 1102. Extension 1144 can be positioned within housing 1110 when tip 1138 is coupled to housing 1110. Cartridge 1102 can be coupled to tip 1138 before tip 1138 is coupled to housing 1110. Tip 1138 can include one or more tip beams 1146. Beam 1146 can be a cantilevered beam. Beam 1146 can extend along a central axis in the longitudinal direction L from a first end to a second end opposite the first end. First end of beam 1146 can be coupled to a body of tip 1138. Second end of beam 1146 can be a free end. A space 1129 can separate beams 1146 from each other. Beams 1146 can be circumferentially spaced from each other. Beams 1146 can be aligned with beams 1130 around an inner perimeter of shell 1108. Beams 1134 of lower body 1114 can fit into the spaces between beams 1146.

Injection device 1100 can be reusable. For example, tip 1138 can be removed from housing 1110 after an injection, a new cartridge 1102 can be loaded into tip 1138, and tip 1138 can be recoupled to housing 1110. A user can grasp upper body 1112 and apply a reloading force to return the injection device to the ready configuration. The reloading force can be longitudinally opposite to the injection force direction. Alternatively, a user could apply the reloading force to plunger 1104 when tip 1138 is detached from housing 1110 to move upper body 1112 and lower body 1114 to the pre-injection configuration.

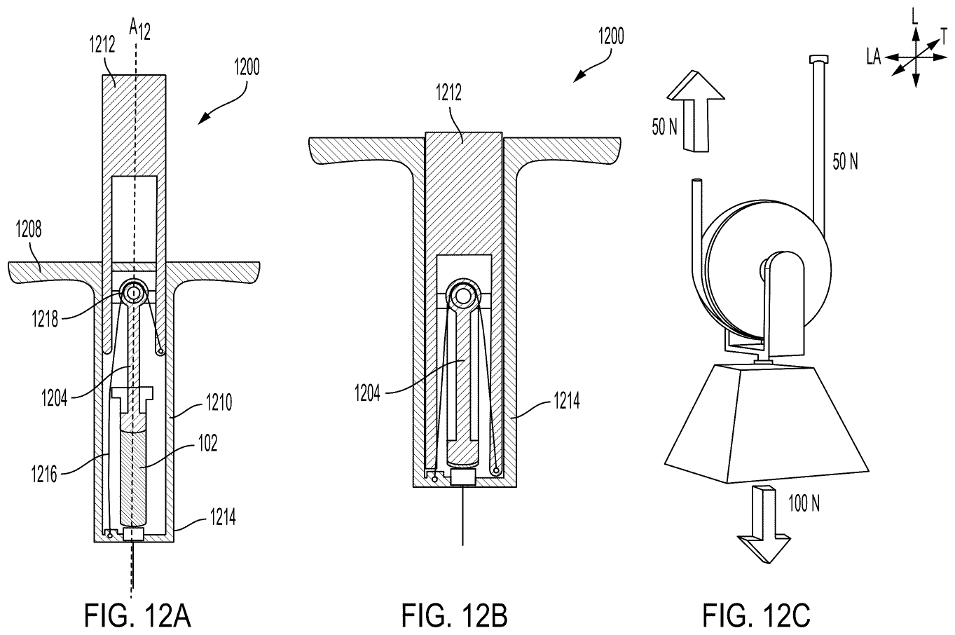

FIGS. 12A-12B illustrate another embodiment of an injection device 1200. Injection device 1200 can be a power-assisted injection device for administering a liquid medication. Injection device 1200 can be adapted to provide a mechanical advantage such that an injection force applied to medicament in a cartridge is greater than a force applied by a user. Injection device 1200 can include a housing 1210 adapted to receive syringe 102. Housing 1210 can include a first end and a second end spaced from the first end along a central axis A₁₂. Central axis A₁₂ can extend in a longitudinal direction L. A lateral direction LA can be perpendicular to longitudinal direction L. A transverse direction T can be perpendicular to each of lateral direction LA and longitudinal direction L. Housing can include a finger flange 1208 engageable by a user during an injection.

Housing 1210 can include an upper body 1212 and a lower body 1214. Upper body 1212 can be movable relative to lower body 1214. Upper body 1212 can be translate longitudinally relative to lower body 1214. One of upper body 1212 and lower body 1214 can be telescopically nested within the other of upper body 1212 and lower body 1214. In some examples, upper body 1212 extends from the proximal end of lower body 1214 when the injection device 1200 is in a post-injection configuration. A tether 1216 can be coupled to upper body 1212 and lower body 1214. Tether 1216 can be a rope, chain, cable, wire, string, or belt. Tether 1216 can include a first end coupled to upper body 1212 and a second end coupled to lower body 1214. Tether 1216 can be coupled to a distal end of upper body 1212. Tether 1216 can be coupled to a distal end of lower body 1214. Distal end of upper body 1212 can engage distal end of lower body 1214 when injection device 1200 is in a post-injection configuration (FIG. 12B).

A plunger 1204 can moveable relative to syringe 102. Plunger 1204 be adapted to dispense liquid medication from a barrel of syringe 102 as plunger 1204 moves relative to syringe 102. Plunger 1204 can include a pulley assembly 1218. Pulley assembly 1218 can include a pulley wheel. Tether 1216 can be engaged with pulley wheel. Pulley assembly 1218 can be positioned proximally of distal portions of each of upper body 1212 and lower body 1214. Plunger 1204 can be movable relative to each of upper body 1212 and lower body 1214.

Upper body 1212 can move relative to lower body 1214 in response to application of a force to upper body 1212. For example, a user can engage finger flange 1208 with their fingers and apply a linear force to upper body 1212 with their thumb. Movement of upper body 1212 relative to lower body 1214 can cause tension in tether 1216. The tension in tether can apply an injection force on plunger 1204 via pulley wheel. The injector 1200 can provide a mechanical advantage to increase the injection force compared to the linear force applied by the user. The injection force on the pulley wheel can be twice that of the linear force as both ends of the line act in cooperation and each end applies a line force equal to the linear force. Upper body 1212 can move longitudinally relative to lower body 1214 by a first amount. Plunger 1204 can move longitudinally relative to lower body 1214 by a second amount. First amount can be greater than second amount.

FIG. 12C illustrates the principle of mechanical advantage provided by a pulley. A line tensioned with 50 N of force provides a force of 100 N to pulley.

Injection device 1200 can be reusable. A user can disengage upper body from lower body 1214. Syringe 102 can be removed from housing 1210 and replaced. Upper body 1212 can then be coupled to lower body 1214 such that injection device 1200 is ready for use.

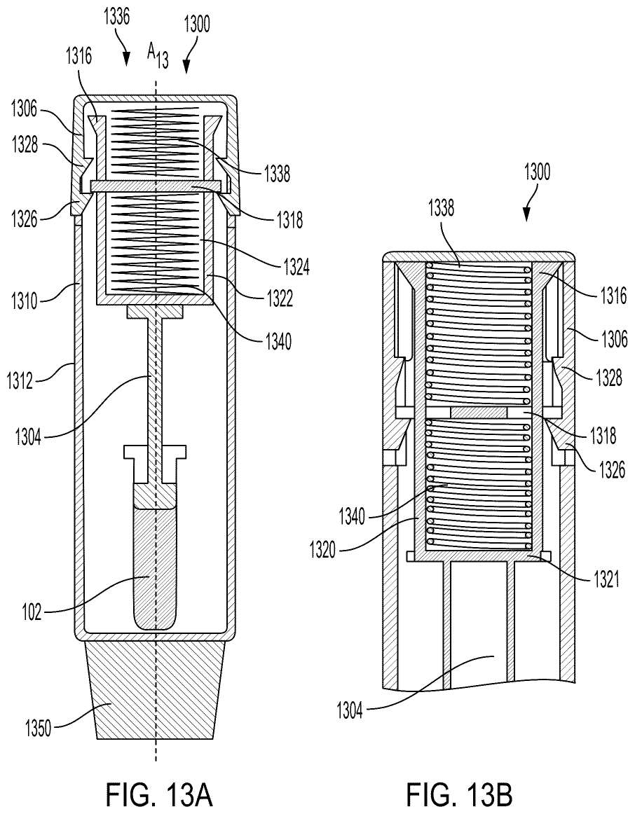

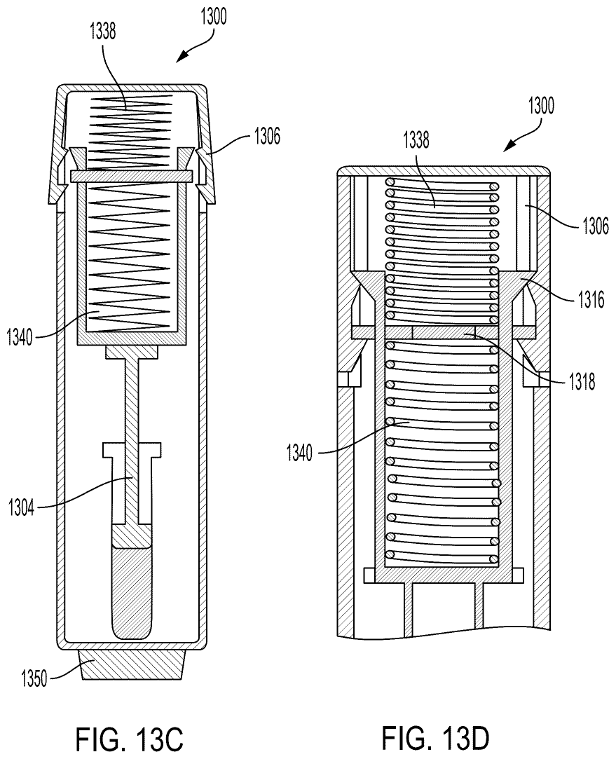

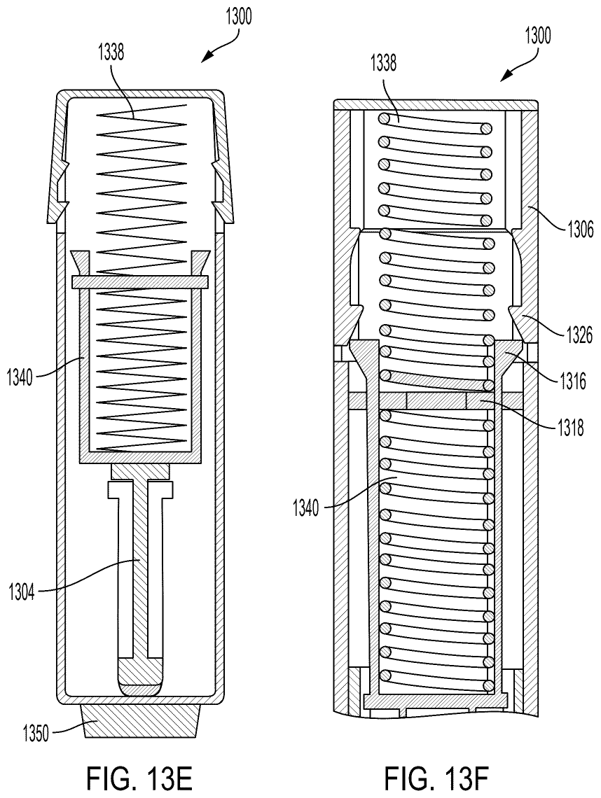

FIGS. 13A-13F illustrate another embodiment of an injection device 1300. Injection device 1300 can be a power-assisted injection device for administering a liquid medication. Injection device 1300 can transition between a ready configuration (FIGS. 13A-13B), a first firing configuration (FIGS. 13C-13D), and a second firing configuration (FIGS. 13E-13F). Injection device 1300 can be adapted to provide a variable injection force during an injection. For example, injection device 1300 can provide a first injection force during a first stage of an injection and a second injection force during a second stage of an injection. The second force can be greater than the first force.

Injection device 1300 can include a housing 1310 with a housing body 1312 adapted to receive syringe 102. Housing 1310 can include a first end and a second end spaced from the first end along a central axis A₁₃. Central axis A₁₃ can extend in a longitudinal direction L. A lateral direction LA can be perpendicular to longitudinal direction L. A transverse direction T can be perpendicular to each of lateral direction LA and longitudinal direction L. Syringe 102 can be fixed relative to housing 1310. A plunger 1304 can be movable relative to syringe 102 to dispense medicament from a needle connected to syringe. Plunger 1304 can form a liquid seal with a sidewall of the barrel.

An inner frame 1320 can be coupled to plunger 1304. Injection device 1300 can be adapted to apply an injection force to inner frame 1320 such that inner frame 1320 moves plunger 1304 relative to housing 1310. Inner frame 1320 can be fixed to plunger 1304. Alternatively, inner frame 1320 can be spaced from each other and move into engagement during an injection. Inner frame 1320 can be adapted to receive a power source to move inner frame 1320 relative to housing body 1312. Inner frame 1320 can include a sidewall 1322 that defines a channel 1324 to receive a motive force device.

Inner frame 1320 can be adapted to receive a first flange 1318. First flange 1318 can extend across a lateral width of inner frame 1320. First flange 1318 can divide channel 1324 of inner frame 1320 into a first channel and a second channel. First flange 1318 can be movable relative to inner frame 1320. First flange 1318 can move longitudinally relative to inner frame 1320. Inner frame 1320 can translate longitudinally relative to housing body 1312 while first flange 1318 remains longitudinally fixed relative to housing body 1312.

An end wall 1321 can be coupled to inner frame 1320. End wall 1321 can be fixed to inner frame 1320 such that end wall 1321 moves relative to housing body 1312 with inner frame 1320. End wall 1321 can be fixed to sidewall 1322. End wall 1321, sidewall 1322, and plunger 1304 can be a unitary construct.

Injection device 1300 can include a latch 1306 adapted to define a locked position that inhibits movement of plunger 1304 and a released position that enables movement of plunger 1304. Latch 1306 can be moveable relative to housing body 1312. Latch 1306 can be movable relative to housing body 1312 from a locked position (FIG. 13A) to an unlocked position (FIG. 13C). Latch 1306 can be a cantilevered beam that extends from housing body 1312. Latch 1306 can flex relative to housing body 1312 from the locked position to the unlocked position. Latch 1306 can flex laterally outwardly relative to housing body 1312 from the locked position to the unlocked position.

Injection device 1300 can include a needle guard 1350 movable relative to housing between an extended position (FIG. 13A) and a retracted position (FIG. 13C). Needle guard 1350 can be configured to at least partially surround a needle when the needle guard 1350 is in the extended position. The needle guard 1350 can envelope at least a portion of the syringe 102 in the extended position. The needle guard 1350 can extend distally beyond a distal end of the needle when the needle guard 1350 is in the extended position. At least a portion of the needle guard 1350 can extend into the housing 1310. Movement of the needle guard 1350 can be configured to activate the injection device 1300. A proximal end (not shown) of the needle guard 1350 can move the latch 1306 as the needle guard 1350 moves from the extended position to the retracted position.

Latch 1306 can include a first tooth 1326 adapted to engage first flange 1318. First tooth 1326 can prevent first flange 1318 from moving distally in a longitudinal direction relative to housing body 1312 when latch 1306 is in the locked position. Latch 1306 can include a second tooth 1328 adapted to engage a second flange 1316. Second flange 1316 can be coupled to inner frame 1320. Second flange 1316 can be coupled to sidewall 1322 of inner frame 1320. Second flange 1316 can be coupled to a proximal end of sidewall opposite end wall 1321.

Second flange 1316 can be adapted to engage second tooth 1328 to move latch 1306 from the locked position to the released position. One of second flange 1316 and second tooth 1328 can include an angled surface such that axial movement of second flange 1316 relative to second tooth 1328 causes latch 1306 to move laterally outward.

First flange 1318 can be engaged with first tooth 1326 when second flange 1316 begins to engage second tooth 1328. First flange 1318 can disengage from first tooth 1326 as latch 1306 moves to the released position. Movement of latch 1306 radially laterally outward can allow first flange 1318 to disengage from first tooth 1326.

Injection device 1300 can include a first latch and latch 1306 can be a second latch. First latch can be moveable from a locked position to an unlocked position. Movement of the first latch can engage actuation of the first power source 1338 to begin the injection. Movement of the first latch can allow inner frame 1320 to move relative to housing body 1312 while latch 1306 is in the locked position.

Injection device 1300 can include a motive force device 1336 for applying an injection force to plunger 1304. Injection device 1300 can be adapted for administering the liquid medication upon release of latch 1306 from the locked position at least in part via motive force device 1336. Motive force device 1336 can include a first power source 1338 and a second power source 1340. First power source 1338 and second power source 1340 can each be positioned within channel 1324. First power source 1338 can be positioned in first portion of channel 1324 and second power source 1340 can be positioned in second portion of cavity with first flange 1318 separating the first and second portions of cavity.

First power source 1338 can be in compression between first flange 1318 and end wall 1321. Second power source 1340 can be in compression between first flange 1318 and housing body 1312. The second power source 1340 can apply a force greater than the force applied by the first power source 1338. The second power source 1340 apply a greater force to overcome any pressure at the injection site as a result of medication that has been delivered to the injection site.

First power source 1338 can provide a first injection force during the first stage of an injection. Second power source 1340 can provide a second injection force during the second stage of an injection. First power source 1338 and second power source 1340 can each be a spring. First power source 1338 and second power source 1340 can be arranged in series such that first power source 1338 provide an injection force during the first stage of an injection and the second power source 1340 provides the injection force during the second stage of the injection. In other embodiments, first power source 1338 can provide power during the first stage of the injection while the first power source 1338 and the second power source 1340 both contribute to the injection force during the second stage of the injection. In still other embodiments, the first stage overlaps the second stage such that first power source 1338 applies the injection force, then both first power source 1338 and second power source 1340 apply the injection force, then second power source 1340 applies the injection force on its own. First power source 1338 and second power source 1340 can be adapted for actuation at least partly in series. For example, the injection force provided by each of first power source 1338 and second power source 1340 can be applied not wholly simultaneously.

The first stage of the injection can begin as the plunger 1304 begins to move relative to housing body 1312. The second stage of the injection can begin when the second flange 1316 engages second tooth 1328 and transitions latch 1306 to the released position. First flange 1318 can be fixed relative to housing body 1312 during the first stage and movable relative to housing body 1312 in a longitudinal direction during the second stage.

Second flange 1316 can engage latch 1306 to lock inner frame 1320 in the second position after an injection. Second flange 1316 can move distally in longitudinal direction L during an injection such that a proximal end of second flange 1316 is positioned distally of a distal end of latch 1306 (FIG. 13F). Latch 1306 can be adapted to resiliently flex toward central axis A₁₃ after second flange 1316 moves past first tooth 1326. Distal end of latch 1306 can engage proximal end of second flange 1316 to inhibit or prevent proximal movement of inner frame 1320 to lock inner frame 1320 in the second position.

It will be appreciated by those skilled in the art that changes could be made to the embodiments described above without departing from the broad inventive concept thereof. Furthermore, it should be appreciated that the structure, features, and methods as described above with respect to any of the embodiments described herein can be incorporated into any of the other embodiments described herein unless otherwise indicated. It is understood, therefore, that this invention is not limited to the particular embodiments disclosed, but it is intended to cover modifications within the spirit and scope of the present disclosure. Further, it should be appreciated, that the term substantially indicates that certain directional components are not absolutely perpendicular to each other and that substantially perpendicular means that the direction has a primary directional component that is perpendicular to another direction.

1. A power-assisted injection device for administering a liquid medication, the injection device comprising:
a syringe including a plunger and a barrel adapted for holding the liquid medication;
a housing including a body adapted for holding the syringe and a latch configured to define a locked position that inhibits movement of the plunger and a released position that enables movement of the plunger, the housing including a first end and a second end opposite the first end along a central axis, the plunger configured to move in a first direction toward the second end to administer the medication;
a syringe holder configured to couple to the barrel;
a needle guard configured to move axially relative to the syringe holder to transition between an initial position in which the distal end of the discharge port extends out of the needle guard, and a retracted position in which the distal end of the discharge port is retracted into the needle guard; and
a motive force device coupled to the second end of the housing, the motive force device configured for applying an injection force to the plunger to administer the medication while the latch is in the released position;
whereby the injection device is configured for administering the liquid medication upon release of the latch from the locked position at least in part via the motive force device applying the injection force to the plunger of the syringe.

2. The injection device of claim 1 wherein the motive force device is at least one spring engaged with the body of the housing.

3. The injection device of claim 2 wherein the housing includes a flange configured to translate relative to the housing body and configured to contact the plunger to apply the injection force to the syringe.

4. The injection device of claim 3 wherein the housing includes a base proximate an upper portion of the barrel of the syringe, the at least one spring of the motive force device is a pair of springs in tension between the housing base and the housing flange.

5. The injection device of claim of claim 4 wherein the latch is a protrusion within the housing, the protrusion contacting an underside of the housing flange when the latch is in the locked position, thereby retaining the housing flange in the locked position until release.

6. The injection device of claim 1, wherein the motive force device is a spring that is coaxial with the plunger.

7. (canceled)

8. (canceled)

9. (canceled)

10. (canceled)

11. (canceled)

12. The injection device of claim 1 wherein the motive force device is a pair of magnetic components, each one of the magnetic components being a magnet and/or a ferrous structure, whereby attraction between the pair of magnetic components applies the injection force to the plunger.

13. The injection device of claim 12 wherein a first one of the pair of magnetic components is fixed relative to the housing proximate a barrel of the syringe and second one of the pair of the magnetic components is coupled to the plunger at or near a plunger flange.

14. The injection device of claim 13 wherein the first one of the pair of magnetic components is a first magnet and the second one of the pair of magnetic components is a second magnet oriented to attract the first magnet.

15. The injection device of claim 13 where the magnets are spaced apart until the completion of the administration of the medication.

16. A power-assisted injection device for administering a liquid medication, the injection device comprising:
a syringe including a plunger and a barrel adapted for holding the liquid medication;
a housing including a body adapted for holding the syringe and a latch configured to define a locked position that inhibits movement of the plunger and a released position that enables movement of the plunger; and
a motive force device configured for applying an injection force to the plunger to administer the medication while the latch is in the released position;
whereby the injection device is configured for administering the liquid medication upon release of the latch from the locked position at least in part via the motive force device applying the injection force to the plunger of the syringe,
wherein the motive force device is a first spring and a second spring, the first spring and the second spring being configured for actuation at least partly in series.

17. The injection device of claim 16 wherein the housing includes a divider separating the first spring from the second spring, the divider configured for movement relative to the housing body.

18. The injection device of claim 17 wherein the latch is a first and second latch, wherein release of the first latch engages actuation of the first spring and release of the second latch engages actuation of the second spring.

19. The injection device of claim 18 wherein the housing further includes an inner frame configured for engage the plunger, the inner frame being moveable relative to the housing body and the divider, wherein upon release of the first latch and before release of the second latch, the first spring moves from a compressed position to an extended position, and upon release of the second latch the second spring moves from a compressed position to an extended position.

20. The injection device of claim 3, wherein the translatable housing flange includes a contact portion configured for contact by a user to move the housing flange upwardly to reset the motive force device.

21. A power-assisted injection device for administering a liquid medication, the injection device comprising:
a syringe including a plunger and a barrel that is configured for holding the liquid medication;
at least one spring configured for applying an injection force to the plunger to administer the medication;
a housing including a body configured for holding the syringe and a flange configured to translate relative to the housing body and configured to contact the plunger to apply the injection force to the syringe; and
a load actuator configured for loading the spring;
whereby the injection device is configured for administering the liquid medication upon loading of the spring.

22. The injection device of claim 21 wherein the load actuator includes a linkage assembly configured to move the at least one spring from a fired position to a ready position.

23. The injection device of claim 22 wherein the linkage assembly includes a lever arm assembly including a lever arm and a pivot fixed on the body of the housing.

24. The injection device of claim 23 wherein the linkage assembly further includes at least one linkage arm pivotally coupled to an interior end of the lever arm, whereby applying a force to the lever arm transmits a loading force through the linkage arm upward to compress the at least one spring.

25. The injection device of claim 23 wherein (i) the linkage assembly further includes a linkage arm pivotally coupled to an interior end of the lever arm, (ii) the at least one spring is a pair of springs engaged with opposing ends of the housing flange, and (iii) the at least one linkage arm is a pair of linkage arms engaged with opposing ends of the housing flange, whereby applying a force to the lever arm transmits a loading force through the linkage arms upward to compress the springs.

26. The injection device of claim 22 wherein spring is a coaxial spring that disposed about the plunger.

27-44. (canceled)
